SEQUENCE LISTING (34 sequences)

SEQ ID NO: 1 - RNA, 21 nt, Artificial Sequence
  Description of Artificial Sequence Synthetic oligonucleotide
acugguacaaggguugggaga

SEQ ID NO: 2 - RNA, 21 nt, Artificial Sequence
  Description of Artificial Sequence Synthetic oligonucleotide
ggcauaggaugacaaagggaa

SEQ ID NO: 3 - RNA, 21 nt, Artificial Sequence
  Description of Artificial Sequence Synthetic oligonucleotide
ccagcuaacaauacacugcca

SEQ ID NO: 4 - RNA, 21 nt, Artificial Sequence
  Description of Artificial Sequence Synthetic oligonucleotide
auuaggaacacaucgcaaaaa

SEQ ID NO: 5 - RNA, 21 nt, Artificial Sequence
  Description of Artificial Sequence Synthetic oligonucleotide
uacuccaaaaggcuacaauca

SEQ ID NO: 6 - RNA, 21 nt, Artificial Sequence
  Description of Artificial Sequence Synthetic oligonucleotide
gccuucugacuccaaguccag

SEQ ID NO: 7 - RNA, 21 nt, Artificial Sequence
  Description of Artificial Sequence Synthetic oligonucleotide
ggcgaaggaugacaaagggaa

SEQ ID NO: 8 - RNA, 21 nt, Artificial Sequence
  Description of Artificial Sequence Synthetic oligonucleotide
cggaagggcagagagggccag

SEQ ID NO: 9 - RNA, 21 nt, Artificial Sequence
  Description of Artificial Sequence Synthetic oligonucleotide
uaccccuggagauucugauaa

SEQ ID NO: 10 - RNA, 21 nt, Artificial Sequence
  Description of Artificial Sequence Synthetic oligonucleotide
cagaccgagacaagugcaaug

SEQ ID NO: 11 - RNA, 21 nt, Artificial Sequence
  Description of Artificial Sequence Synthetic oligonucleotide
aucagcuaaugacacugccua

SEQ ID NO: 12 - RNA, 20 nt, Artificial Sequence
  Description of Artificial Sequence Synthetic oligonucleotide
gcucaauaaauaccguugaa

SEQ ID NO: 13 - RNA, 21 nt, Artificial Sequence
  Description of Artificial Sequence Synthetic oligonucleotide
gcaaggucgguucuacgggug

SEQ ID NO: 14 - RNA, 21 nt, Artificial Sequence
  Description of Artificial Sequence Synthetic oligonucleotide
accugcacuguaagcacuuug

SEQ ID NO: 15 - RNA, 21 nt, Artificial Sequence
  Description of Artificial Sequence Synthetic oligonucleotide
gaaggcaacacggauaaccua

SEQ ID NO: 16 - RNA, 22 nt, Artificial Sequence
  Description of Artificial Sequence Synthetic oligonucleotide
ucucccaacccuuguaccagug

SEQ ID NO: 17 - RNA, 22 nt, Artificial Sequence
  Description of Artificial Sequence Synthetic oligonucleotide
uucccuuugucauccuaugccu

SEQ ID NO: 18 - RNA, 22 nt, Artificial Sequence
  Description of Artificial Sequence Synthetic oligonucleotide
uggcaguguauuguuagcuggu

SEQ ID NO: 19 - RNA, 22 nt, Artificial Sequence
  Description of Artificial Sequence Synthetic oligonucleotide
uuuuugcgauguguuccuaaua

SEQ ID NO: 20 - RNA, 23 nt, Artificial Sequence
  Description of Artificial Sequence Synthetic oligonucleotide
ugauuguagccuuuuggaguaga

SEQ ID NO: 21 - RNA, 22 nt, Artificial Sequence
  Description of Artificial Sequence Synthetic oligonucleotide
cuggacuuggagucagaaggcc

SEQ ID NO: 22 - RNA, 22 nt, Artificial Sequence
  Description of Artificial Sequence Synthetic oligonucleotide
uucccuuugucauccuucgccu

SEQ ID NO: 23 - RNA, 22 nt, Artificial Sequence
  Description of Artificial Sequence Synthetic oligonucleotide
cuggcccucucugcccuuccgu

SEQ ID NO: 24 - RNA, 22 nt, Artificial Sequence
  Description of Artificial Sequence Synthetic oligonucleotide
uuaucagaaucuccagggguac

SEQ ID NO: 25 - RNA, 22 nt, Artificial Sequence
  Description of Artificial Sequence Synthetic oligonucleotide
cauugcacuugucucggucuga

SEQ ID NO: 26 - RNA, 23 nt, Artificial Sequence
  Description of Artificial Sequence Synthetic oligonucleotide
uaggcagugucauuagcugauug

SEQ ID NO: 27 - RNA, 22 nt, Artificial Sequence
  Description of Artificial Sequence Synthetic oligonucleotide
uucaacggguauuuauugagca

SEQ ID NO: 28 - RNA, 22 nt, Artificial Sequence
  Description of Artificial Sequence Synthetic oligonucleotide
cacccguagaaccgaccuugcg

SEQ ID NO: 29 - RNA, 24 nt, Artificial Sequence
  Description of Artificial Sequence Synthetic oligonucleotide
caaagugcuuacagugcagguagu

SEQ ID NO: 30 - RNA, 22 nt, Artificial Sequence
  Description of Artificial Sequence Synthetic oligonucleotide
uagguuauccguguugccuucg

SEQ ID NO: 31 - DNA, 22 bp, Artificial Sequence
  Description of DNA/RNA hybrid molecule Synthetic oligonucleotide
acugguacaaggguugggagat

SEQ ID NO: 32 - DNA, 17 bp, Artificial Sequence
  Description of DNA/RNA hybrid molecule Synthetic oligonucleotide
uacaaggguugggagat

SEQ ID NO: 33 - DNA, 17 bp, Artificial Sequence
  Description of DNA/RNA hybrid molecule Synthetic oligonucleotide
uacaaggguugggagat

SEQ ID NO: 34 - DNA, 17 bp, Artificial Sequence
  Description of DNA/RNA hybrid molecule Synthetic oligonucleotide
uacaaggguugggagat

(19) United States
(12) Patent Application Publication
(10) Pub. No.: US 2009/0105174 A1
(43) Pub. Date: Apr. 23, 2009

(54) NUCLEIC ACIDS HYBRIDIZABLE TO MICRO RNA AND PRECURSORS THEREOF

(75) Inventor: Sumedha D. Jayasena, Thousand Oaks, CA (US)

Correspondence Address: AMGEN INC.;LAW DEPARTMENT, 1201 AMGEN COURT WEST, SEATTLE, WA, 98119, US

(73) Assignee: AMGEN INC., Thousand Oaks, CA (US)

(21) Appl. No.: 12/105,562

(22) Filed: Apr. 18, 2008

Related U.S. Application Data
(60) Provisional application No. 60/913,200, filed on Apr. 20, 2007.

(51) Int. Cl. A61K 31/7105; C12N 5/06; C07H 21/02; C12N 5/08

(52) U.S. Cl. 514/44; 435/375; 435/366; 536/23.1

(57) ABSTRACT

Methods and compositions relating to nucleic acids targeting certain miRNA molecules are disclosed. The nucleic acids are useful in methods of increasing nuclear concentration of FKHR protein, decreasing cell viability, and treating cancer.

### CROSS-REFERENCES TO RELATED APPLICATIONS

This application claims priority to U.S. Provisional Patent Application No. 60/913,200, filed Apr. 20, 2007, which is incorporated herein by reference in its entirety for all purposes.

### BACKGROUND

MicroRNAs (miRNAs) regulate gene expression through an RNA interference (RNAi) mechanism by targeting specific messages and inhibiting their translation. The genes encoding miRNAs are longer than the processed miRNA molecule. miRNAs are first transcribed as primary transcripts or pri-miRNA and processed to short, approximately 70-nucleotide stem-loop structures known as pre-miRNA in the cell nucleus. This processing is performed in humans by a protein complex known as the Microprocessor complex, including the nuclease Drosha and the double-stranded RNA binding protein DGCR8. These pre-miRNAs are then processed to mature miRNAs in the cytoplasm by interaction with the endonuclease Dicer assisted by TRBP, which also initiates the formation of the RNA-induced silencing complex (RISC). This complex is responsible for the gene silencing observed due to miRNA expression and RNA interference. The pathway in plants varies slightly due to their lack of Drosha homologs. Instead, Dicer homologs alone effect several processing steps.

Efficient processing of pri-miRNA by Drosha requires the presence of extended single-stranded RNA on both 3′- and 5′-ends of a hairpin molecule. The Drosha complex cleaves RNA molecules at approximately two helical turns away from the terminal loop and approximately one turn away from basal segments. In most analyzed molecules this region contains unpaired nucleotides and the free energy of the duplex is relatively high compared to lower and upper stem regions. The resulting pre-miRNA has a short hairpin loop structure and is exported to the cytoplasm by Exportin 5 with the help from a cofactor Ran, a GTPase (Gwizdek et al., J Biol Chem 278, 5505-8 (2003); Lund et al., Science 303, 95-8 (2004); Bohnsack et al., RNA 10, 185-91 (2004)).

When Dicer cleaves the pre-miRNA stem-loop in the cytoplasm, two complementary short RNA molecules are formed, but only one is integrated into the RISC complex on the basis of the stability of the 5′ end. The remaining strand, known as the passenger strand is degraded. After integration into the active RISC complex, miRNAs base pair with their complementary mRNA molecules and induce down regulation of the expression of the transcript by one of the two key mechanisms, depending on the degree of complementarity between the miRNA and the target mRNA. In animals, pairing between miRNA and their target mRNAs is not usually perfect, although there are few exceptions where perfect or near perfect recognition exist (Yekta et al., Science 304, 594-6 (2004); Mansfield et al. Nat Genet. 36, 1079-83 (2004)). If the complementarity between the miRNA and the target is perfect or near perfect, then the cleavage of the mRNA is mediated by the endonuclease (slicer) activity in the RISC provided by Ago2 protein. Where miRNAs bind to their targets via imperfect base pairing, miRNA bound messages may be directed to a cytoplasmic foci known as P-bodies or processing bodies where the ribosomes are depleted but rich in nucleases (Parker et al., Nature Structural & Molecular Biology 11, 121-7 (2004)). P-bodies serve as either degradation centers or storage depots for these messages, where their translation is inhibited.

To date, close to 500 miRNAs have been identified in humans (Griffiths-Jones, S, Nucleic Acids Res 32, D109-11 (2004)). Bioinformatics approaches have predicted that these miRNAs are capable of regulating at least 30% of human transcripts (Lewis, et al. Cell, 2005.120(1): p. 15-20). As a result, miRNAs have the potential to play a vital role in many biological processes whose deregulation could lead to various disease states. Experimental evidence is accumulating to point out their roles in many biological processes. These attributes make miRNAs a potential class of targets for therapeutic intervention. However, the lack of current understanding on specific roles played by individual miRNAs in a plethora of biological processes has complicated the targeting of miRNAs.

The forkhead homolog of rhabdomyosarcoma protein (also known as the forkhead transcription factor FKHR or FOX01; GeneID: 2308) is a transcription factor downstream of the P13-kinase/AKT pathway. The latter is activated in about 30% of human tumors and represents an important pathway for therapeutic intervention in oncology. A common mechanism of P13K activation is through the loss of PTEN tumor suppressor activity. Activated P13K leads to the activation of AKT, a serine threonine kinase, which in turn inhibits apoptosis and promotes cell survival. The activated AKT phosphorylates FKHR and induces nuclear export. Thus, methods of increasing FKHR concentration in the nucleus would be useful in decreasing cell viability (e.g. increasing cell apoptosis), and thus helpful in therapeutic oncology.

The methods and compositions described herein address these and other needs in the art.

### BRIEF DESCRIPTION OF THE DRAWINGS

FIG. 1 illustrates an example of data obtained from experiments, presented as a plot of the ratio of FKHR concentration in the nucleus to FKHR concentration in the cytoplasm versus cell density (an indicator of cell death).

FIG. 2 illustrates a plot of the ratio of FKHR concentration in the nucleus to FKHR concentration in the cytoplasm at nucleic acid concentrations of 2.5 nM and 10 nM.

FIG. 3 illustrates examples of target regions of miRNA precursors.

FIG. 4 illustrates cellular images of MDA-468 cells bearing GFP-FKHR fusion protein 48 hrs after transfection of LNA inhibitors to selected miRNAs: (A) untreated; (B) mock (lipofectin treated only) (C) miRNA-16; (D) miRNA-204; (E) miRNA-508; and (F) Strausporin.

FIG. 5 illustrates results from cell viability study of MiaPaCa2 cells 72 hrs after transfection of LNA inhibitors: Panel A illustrates results using nucleic acids targeting miRNA-150, miRNA-204, and miRNA-449; and Panel B illustrates results using nucleic acids targeting miRNA-450-1, miRNA-508, and a negative control nucleic acid targeting miRNA-16.

FIG. 6 illustrates results from cell viability study of T24 cells 72 hrs after transfection of LNA inhibitors: Panel A illustrates results using nucleic acids targeting miRNA-150, miRNA-204, and miRNA-449; and Panel B illustrates results using nucleic acids targeting miRNA-450-1, miRNA-508, and a negative control nucleic acid targeting miRNA-16.

FIG. 7 illustrates results from cell viability study of HeLa cells 72 hrs after transfection of LNA inhibitors: Panel A illustrates results using nucleic acids targeting miRNA-150, miRNA-204, and miRNA-449, and miRNA-508; and Panel B illustrates results using nucleic acids targeting miRNA-204, miRNA-508, miRNA-449, and a negative control nucleic acid targeting miRNA-16.

FIG. 8 illustrates the results of different chemically modified anti-miRNA nucleic acids as a plot of the ratio of nuclear FKHR to cytoplasmic FKHR (AVG NC-PP) versus cell density (AVG Cell_PP), where the integer 1 denotes LNA anti-miRNA nucleic acids as set forth in Example 4, integer 2 denotes LNA/DNA anti-miRNA nucleic acids as set forth in Example 4, integer 3 denotes OMe/DNA anti-miRNA nucleic acids as set forth in Example 4, and integer 4 denotes LNA/OMe anti-miRNA nucleic acids as set forth in Example 4.

FIG. 9 illustrates the uptake of nucleic acid sequences conjugated to cholesterol and fluorescein by different adherent cells. The amount of intracellular fluorescence is shown as a function of the concentration of nucleic acid conjugated to fluorescein and cholesterol.

FIG. 10 illustrates cell cycle analysis of cells treated with nucleic acid inhibitors with (left) or without (right) cholesterol conjugation. The percentage of G2/M cell population in each case is shown.

FIG. 11 illustrates an analysis of caspase 3 levels in cells treated with nucleic acid inhibitors with (left) or without (right) cholesterol conjugation. The percentage of Caspase-3 levels in each case is shown.

### SUMMARY

It has been discovered that nucleic acid sequences designed to hybridize to certain miRNAs and precursors thereof are useful in increasing the nuclear concentration of forkhead homolog of rhabdomyosarcoma (FKHR), decreasing cell viability (e.g. increasing apoptosis and/or decreasing cellular proliferation), and treating cancer.

In one aspect, a method is provided for increasing the concentration of a FKHR protein within the nucleus of a cell. The method includes introducing into the cell a nucleic acid that is hybridizable to an RNA molecule, is antisense to an RNA molecule, is substantially complimentary to an RNA molecule, and/or has a sequence with at least 70% sequence identity to a 6 nucleobase (or nucleotide) sequence (i.e. contiguous sequence) within one of SEQ ID NOs: 1-15 (also referred to herein as “anti-miRNA nucleic acid sequences”). The RNA molecule is an miRNA selected from miRNA-17-5p, miRNA-25, miRNA-34b, miRNA-95, miRNA-99b, miRNA-150, miRNA-154, miRNA-204, miRNA-211, miRNA-328, miRNA-361, miRNA-422b, miRNA-449, miRNA-450-1, miRNA-508, and precursors thereof.

In another aspect, a method is provided for decreasing cell viability (e.g. increasing apoptosis and/or decreasing cellular proliferation) in a cell. The method includes introducing into the cell a nucleic acid that is hybridizable to an RNA molecule, is antisense to an RNA molecule, is substantially complimentary to an RNA molecule, and/or has a sequence with at least 70% sequence identity to a 6 nucleobase (or nucleotide) sequence within one of SEQ ID NOs: 1-15. The RNA molecule is an miRNA selected from miRNA-17-5p, miRNA-25, miRNA-34b, miRNA-95, miRNA-99b, miRNA-150, miRNA-154, miRNA-204, miRNA-211, miRNA-328, miRNA-361, miRNA-422b, miRNA-449, miRNA-450-1, miRNA-508, and precursors thereof.

In another aspect, a method is provided for treating cancer in a subject in need thereof. The method includes administering to the subject an effective amount of a nucleic acid that is hybridizable to an RNA molecule, is antisense to an RNA molecule, is substantially complimentary to an RNA molecule, and/or has a sequence with at least 70% sequence identity to at least a 6 nucleobase (or nucleotide) sequence within one of SEQ ID NOs: 1-15. The RNA molecule is an miRNA selected from miRNA-17-5p, miRNA-25, miRNA-34b, miRNA-95, miRNA-99b, miRNA-150, miRNA-154, miRNA-204, miRNA-211, miRNA-328, miRNA-361, miRNA-422b, miRNA-449, miRNA-450-1, miRNA-508, and precursors thereof.

In another aspect, a nucleic acid is provided having at least 50% locked nucleic acid units. The nucleic acid is hybridizable to an RNA molecule, is antisense to an RNA molecule, is substantially complimentary to an RNA molecule, and/or has a sequence with at least 70% sequence identity to a 6 nucleobase (or nucleotide) sequence within one of SEQ ID NOs: 1-15. The RNA molecule is an miRNA selected from miRNA-17-5p, miRNA-25, miRNA-34b, miRNA-95, miRNA-99b, miRNA-150, miRNA-154, miRNA-204, miRNA-211, miRNA-328, miRNA-361, miRNA-422b, miRNA-449, miRNA-450-1, miRNA-508, and precursors thereof. In some embodiments, the nucleic acid includes at least 60%, 65%, 70%, 75%, 80%, 85%, 90%, or 95% locked nucleic acid units.

In other embodiments, a nucleic acid provided herein further includes at least one cholesterol moiety. In some embodiments a nucleic acid further includes a plurality of cholesterol moieties. The cholesterol moiety or moieties may be linked to the 5′ or 3′, or 5′ and 3′, terminus of the nucleic acid.

### DETAILED DESCRIPTION

### I. Definitions

As used herein, “nucleic acid” means single-, double-, or multiple-stranded DNA, RNA and derivatives thereof. In certain embodiments, the nucleic acid is single stranded. Modifications may include those that provide other chemical groups that incorporate additional charge, polarizability, hydrogen bonding, electrostatic interaction, and functionality to the nucleic acid. Such modifications include, but are not limited to, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping moieties. A 2′deoxy nucleic acid linker is a divalent nucleic acid of any appropriate length and/or internucleotide linkage wherein the nucleotides are 2′deoxy nucleotides. A “nucleobase” refers to the portion(s) of a nucleic acid involved in hybridization (base pairing), and includes, but is not limited to, nitrogenous bases such as cytosine, guanine, adenine, thymine, uracil, and derivatives thereof. A “nucleic acid unit,” as used herein, refers to the portions of a nucleic acid that are linked together by internucleotide linkages, and contain a nucleobase (e.g. a nucleoside).

Certain nucleic aid compounds can exist in unsolvated forms as well as solvated forms, including hydrated forms. In general, the solvated forms are equivalent to unsolvated forms. Certain nucleic aid compounds may exist in multiple crystalline or amorphous forms. In general, all physical forms are equivalent for the uses contemplated by the methods provided herein.

Certain nucleic aid compounds may possess asymmetric carbon atoms (optical centers) or double bonds; the racemates, diastereomers, geometric isomers and individual isomers.

The nucleic aid compounds may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C).

As used herein, the term “miRNA precursor,” or “precursor thereof” in reference to a particular miRNA refers broadly to any precursor which through processing in a cell results in the specified miRNA. The term thus includes the corresponding pri-miRNA, pre-miRNA or variant thereof. In some embodiments, the precursor is the corresponding pri-miRNA or pre-miRNA. The pre-miRNA sequence may include, for example, from 45-90, 60-80 or 60-70 nucleotides. The sequence of the pre-miRNA may include the entire miRNA sequence, or be that of a pri-miRNA excluding from 0-160 nucleotides from the 5′ and 3′ ends of the pri-miRNA. The sequence of the pre-miRNA may comprise the sequence of a hairpin loop. The pri-miRNA sequence may comprise from 45-250, 55-200, 70-150 or 80-100 nucleotides. The sequence of the pri-miRNA may include the pre-miRNA or miRNA as set forth in Table 3 below. The pri-miRNA may also include a hairpin structure (e.g. from 37-50 nucleotides).

The term “hybridization” refers to the pairing of complementary strands of nucleic acids, including triple-stranded nucleic acid hybridization. The mechanism of pairing involves hydrogen bonding, which may be Watson-Crick, Hoogsteen or reversed Hoogsteen hydrogen bonding, between complementary nucleoside or nucleotide bases (nucleobases) of the strands of nucleic acids. For example, adenine and thymine are complementary nucleobases that pair through the formation of hydrogen bonds. Hybridization can occur under varying circumstances.

The phrases “specifically hybridizable” or “hybridizes specifically to” and other similar phrases refer to the association of a nucleic acid with an miRNA, or miRNA precursor, resulting in interference with the normal function of the miRNA, or miRNA precursor (e.g. by altering the activity, disrupting the function, or modulating the level of the miRNA or miRNA precursor). Where a nucleic acid is “specifically hybridizable,” to an miRNA or miRNA precursor, there is a sufficient degree of complementarity to avoid non-specific binding of the nucleic acid to nucleic acid sequences other than the intended miRNA or miRNA precursor under conditions in which specific hybridization is desired (e.g. under physiological conditions in the case of in vivo assays or therapeutic treatment, and under standard assay conditions in the case of in vitro assays). The sequence of the nucleic acid need not be 100% complementary to that of its target miRNA or miRNA precursor to be specifically hybridizable. Moreover, the nucleic acid may hybridize over one or more segments of the miRNA or miRNA precursor such that intervening or adjacent segments are not involved in the hybridization (e.g., a bulge, a loop structure or a hairpin structure).

The term “stringent hybridization conditions” or “stringent conditions” refers to conditions under which a nucleic acid hybridizes to an miRNA or miRNA precursor to form a stable complex (e.g. a duplex), but to a minimal number of other sequences. The stability of complex is a function of salt concentration and temperature (See, for example, Sambrook et al., Molecular Cloning: A Laboratory Manual 2d Ed. (Cold Spring Harbor Laboratory, (1989); incorporated herein by reference). Stringency levels used to hybridize a nucleic acid to an miRNA or miRNA precursor can be readily varied by those of skill in the art. The phrase “low stringency hybridization conditions” refers to conditions equivalent to hybridization in 10% formamide, 5 times Denhart's solution, 6 times SSPE, 0.2% SDS at 42° C., followed by washing in 1 times SSPE, 0.2% SDS, at 50° C. Denhart's solution and SSPE are well known to those of skill in the art as are other suitable hybridization buffers. (See, e.g., Sambrook et al.). The term “moderately stringent hybridization conditions” refers to conditions equivalent to hybridization in 50% formamide, 5 times Denhart's solution, 5 times SSPE, 0.2% SDS at 42° C., followed by washing in 0.2 times SSPE, 0.2% SDS, at 60° C. The term “highly stringent hybridization conditions” refers to conditions equivalent to hybridization in 50% formamide, 5 times Denhart's solution, 5 times SSPE, 0.2% SDS at 42° C., followed by washing in 0.2 times SSPE, 0.2% SDS, at 65° C.

“Complementary,” as used herein, refers to the capacity for precise pairing of two nucleobases (e.g. A to T (or U), and G to C) regardless of where in the nucleic acid or miRNA or miRNA precursor the two are located. For example, if a nucleobases at a certain position of nucleic acid is capable of hydrogen bonding with a nucleobases at a certain position of an miRNA or miRNA precursor, then the position of hydrogen bonding between the nucleic acid and the miRNA or miRNA precursor is considered to be a complementary position. The nucleic acid and miRNA or miRNA precursor are “substantially complementary” to each other when a sufficient number of complementary positions in each molecule are occupied by nucleobases that can hydrogen bond with each other. Thus, the term “substantially complementary” is used to indicate a sufficient degree of precise pairing over a sufficient number of nucleobases such that stable and specific binding occurs between the nucleic acid and an miRNA or miRNA precursor. The phrase “substantially complementary” thus means that there may be one or more mismatches between the nucleic acid and the miRNA or miRNA precursor when they are aligned, provided that stable and specific binding occurs. The term “mismatch” refers to a site at which a nucleobases in the nucleic acid and a nucleobases in the miRNA or precursor with which it is aligned are not complementary. The nucleic acid and miRNA or miRNA precursor are “perfectly complementary” to each other when the nucleic acid is fully complementary to the miRNA or miRNA precursor across the entire length of the nucleic acid.

Generally, a nucleic acid is “antisense” to an miRNA or miRNA precursor when, written in the 5′ to 3′ direction, it comprises the reverse complement of the corresponding region of the target nucleic acid. “Antisense compounds” are also often defined in the art to comprise the further limitation of, once hybridized to a target, being able to modulate levels, expression or function of the target compound.

As used herein, “sequence identity” or “identity” refers to the nucleobases in two sequences that are the same when aligned for maximum correspondence over a specified comparison window. As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “pharmaceutically acceptable salts” is meant to include salts of the active compounds which are prepared with relatively nontoxic acids or bases, depending on the particular substituents found on the compounds described herein. When nucleic aid compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When nucleic aid compounds contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain nucleic aid compounds contain both basic and acidic functionalities that allow the compounds to be converted into either base or acid addition salts.

The neutral forms of the nucleic aid compounds may be regenerated by contacting the salt with a base or acid and isolating the parent compound in the conventional manner. The parent form of the compound differs from the various salt forms in certain physical properties, such as solubility in polar solvents.

In addition to salt forms, nucleic aid compounds are provided which are in a prodrug form. Prodrugs of the nucleic acids described herein are those compounds that readily undergo chemical changes under physiological. Additionally, prodrugs can be converted to the nucleic acids by chemical or biochemical methods in an ex vivo environment. For example, prodrugs can be slowly converted to the nucleic acids when placed in a transdermal patch reservoir with a suitable enzyme or chemical reagent.

The term “treating” refers to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat a cell proliferation disorder, such as cancer, by decreasing the incidence of the disorder and/or causing remission of the disorder.

As used herein, a cell proliferative disorder is, or the proliferating cells are, derived from a cancerous or a non-cancerous cell proliferation disorder. Exemplary cancerous and non-cancerous cell proliferation disorders include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, acute lymphocytic leukemia, lymphocytic leukemia, large granular lymphocytic leukemia, acute myelocytic leukemia, chronic leukemia, polycythemia vera, Hodgkin's lymphoma, non-Hodgkin's lymphoma, multiple myeloma, Waldenstrobm's macroglobulinemia, heavy chain disease, lymphoblastic leukemia, T-cell leukemia, T-lymphocytic leukemia, T-lymphoblastic leukemia, B cell leukemia, B-lymphocytic leukemia, mixed cell leukemias, myeloid leukemias, myelocytic leukemia, myelogenous leukemia, neutrophilic leukemia, eosinophilic leukemia, monocytic leukemia, myelomonocytic leukemia, Naegeli-type myeloid leukemia, nonlymphocytic leukemia, osteosarcoma, promyelocytic leukemia, non-small cell lung cancer, epithelial lung carcinoma, pancreatic carcinoma, pancreatic ductal adenocarcinoma, glioblastoma, metastatic breast cancer, melanoma, and prostate cancer. In certain embodiments, the cell proliferation disorder is osteosarcoma, promyelocytic leukemia, non-small cell lung cancer, epithelial lung carcinoma, pancreatic carcinoma, pancreatic ductal adenocarcinoma, glioblastoma, metastatic breast cancer, melanoma, or prostate cancer.

The term “cancer” refers to all types of cancer, neoplasm or malignant tumors found in mammals, including leukemia, carcinomas and sarcomas. Exemplary cancers include cancer of the breast, brain, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P₃₈₈ leukemia model is widely accepted as being predictive of in vivo anti-leukemic activity. It is believed that a compound that tests positive in the P₃₈₈ assay will generally exhibit some level of anti-leukemic activity in vivo regardless of the type of leukemia being treated. Accordingly, the methods herein include treating leukemia, and methods of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniformi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “antineoplastic” means inhibiting or preventing the growth of cancer. In certain embodiments, the nucleic acids described herein are antineoplastic nucleic acids.

As used herein “combination therapy” or “adjunct therapy” means that the patient in need of the drug is treated or given another drug for the disease in conjunction with the nucleic acid. This combination therapy can be sequential therapy where the patient is treated first with one drug and then the other or the two drugs are given simultaneously.

“Patient” refers to a mammalian subject (e.g. human).

### II. OVERVIEW

Various methods and compositions are provided herein based, in part, upon the identification of certain miRNAs that are involved in decreasing cell viability, regulating the nuclear translocation of a forkhead homolog of rhabdomyosarcoma (FKHR) and involved in the inhibition of cellular apoptosis. Nucleic acids that are capable of hybridizing to these identified miRNAs and thus decreasing cell viability (e.g. increasing apoptosis and/or decreasing cellular proliferation) are useful in the treatment of various cell proliferative disorders, including cancers. The miRNAs found to be involved in decreasing cell viability, including FKHR translocation, include miRNA-17-5p, miRNA-25, miRNA-34b, miRNA-95, miRNA-99b, miRNA-150, miRNA-154, miRNA-204, miRNA-211, miRNA-328, miRNA-361, miRNA-422b, miRNA-449, miRNA-450-1, and miRNA-508.

Disclosed herein are nucleic acids with particular sequences and chemical structure that can hybridize to these miRNAs and thus inhibit their activity. Pharmaceutical compositions containing these nucleic acids are also provided. These nucleic acids and compositions can be used to increase the concentration of FKHR protein within the nucleus of a cell, to decrease cell viability (e.g. increase apoptosis) of a cell, and to treat cancer.

### III. MODULATING NUCLEAR TRANSLOCATION OF FKHR AND DECREASING CELL VIABILITY

Methods for increasing the concentration of a forkhead homolog of rhabdomyosarcoma (FKHR) protein within the nucleus of a cell and decreasing cell viability (e.g. increasing apoptosis and/or decreasing cellular proliferation) in a cell include introducing into the cell a nucleic acid hybridizable to an RNA molecule, such nucleic acid also being referred to herein as an anti-miRNA nucleic acid. The RNA molecule is an miRNA selected from miRNA-17-5p, miRNA-25, miRNA-34b, miRNA-95, miRNA-99b, miRNA-150, miRNA-154, miRNA-204, miRNA-211, miRNA-328, miRNA-361, miRNA-422b, miRNA-449, miRNA-450-1, miRNA-508, and precursors thereof. See Table 3 for the complete sequences of these miRNAs.

The increase in the nuclear concentration of FKHR is relative to the concentration of FKHR in the cell nucleus in the absence of the nucleic acid. Thus, an effective amount of the nucleic acid is introduced to the cell to result in the increase in nuclear concentration of FKHR. Without being bound by any particular theory, the observed increase of FKHR in the nucleus is thought to result from an increase in the nuclear translocation of the FKHR protein from the cytoplasm to the nucleus. Thus, in some embodiments, the methods of increasing the concentration of an FKHR protein within the nucleus of a cell include methods of increasing nuclear translocation of the FKHR protein.

The decrease in cell viability or increase in apoptosis is relative to the incidence of cell death or apoptosis observed in a population of the cell in the absence of the nucleic acid (e.g. more of the cells are induced into the death process as compared to non-exposure to (contact with) the nucleic acid). Thus, an effective amount of the nucleic acid is introduced into the cell to result in an increase in cell death or apoptosis. Apoptosis is generally considered to be a form of cell death in which a controlled sequence of events (or program) leads to the elimination of the cell. Increasing apoptosis also includes the inhibition of cell division that results in a decrease in the total number of viable cancer (e.g. tumor) cells.

The sequence of the nucleic acid may be designed such that it will hybridize to a particular miRNA or miRNA precursor or a region or segment thereof. “Targeting” thus includes determination of at least one target region, segment, or site within the target miRNA or miRNA precursor for the interaction to occur such that the desired effect, e.g., modulation of levels, expression or function, will result. As used herein, the term “region” or “target region” is defined as a portion of the target miRNA or miRNA precursor having at least one identifiable sequence, structure, function, or characteristic.

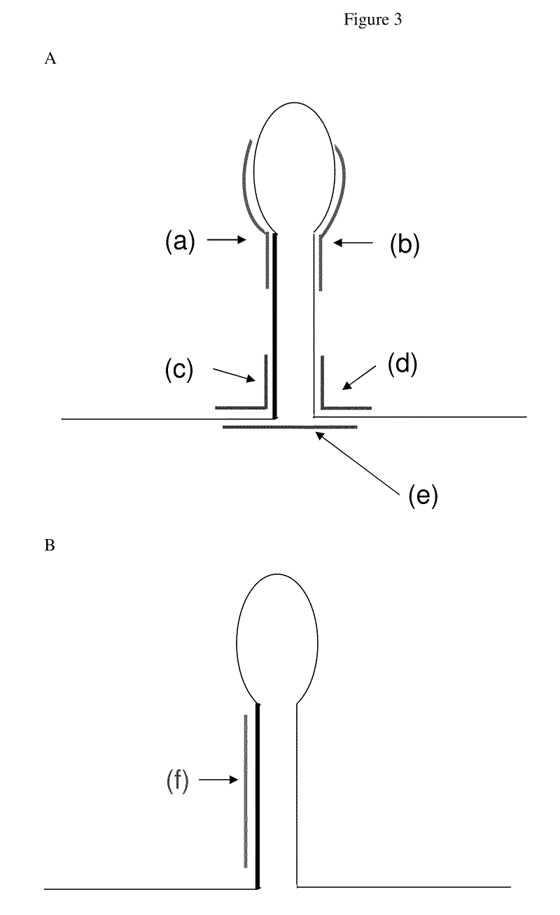

In some embodiments, a nucleic acid is designed to hybridize to a single continuous region of the target miRNA or miRNA precursor. See FIG. 3A and FIG. 3B. In other embodiments, a single nucleic acid is designed to bind to two different regions of a target miRNA or miRNA precursor.

For example, without being bound by any particular theory, a nucleic acid may be designed to block the processing of pre-miRNAs by Dicer by targeting part of the loop and part of the stem of a pre-miRNA (see e.g. nucleic acids (a) and (b) in FIG. 3A). In other embodiments, nucleic acids are designed to block the processing of pri-miRNAs by Drosha by targeting part of the stem and part of either part of the single stranded RNA at the base of the stem (see e.g. nucleic acids (c) and (d) in FIG. 3A). Without being bound by any particular theory, the export of pre-miRNA to the cytoplasm by Exportin may be blocked by targeting pre-miRNA. In some embodiments, a nucleic acid may be designed to block Drosha processing by targeting two discontinuous extensions of the base of the stem in a pri-miRNA sequence (see e.g. nucleic acids (e) in FIG. 3A). In another embodiment, a nucleic acid may be designed to target the stem portion of an miRNA precursor (see e.g. nucleic acid (f) in FIG. 3B). Thus, when a nucleic acid is referred to as being able to hybridize to a miRNA it is meant that the nucleic acid can hybridize, for example, in any of the configurations shown in FIGS. 3A and 3B.

Any portions of the miRNA participating in mRNA binding may be targeted. In some embodiments, the first 6, 7, or 8 nucleotides from the 5′ end of the miRNA may be targeted. Such locations on the target miRNA or precursor thereof to which nucleic acid hybridizes may be referred to as a “suitable target segment.” As used herein, the term “suitable target segment” is defined as at least a 6, 7 or 8-nucleotide portion of a target region to which a nucleic acid is targeted. Once one or more target regions have been identified, nucleic acids are designed to be sufficiently complementary to the target, i.e., hybridize sufficiently well and with sufficient specificity, to give the desired effect (e.g. anti-cancer effect, increasing apoptosis, and or increasing nuclear concentration of FKHR protein).

In some embodiments, the one or more anti-miRNA nucleic acid may be targeted to a first miRNA target and one or more additional anti-miRNA nucleic acids targeted to a second miRNA target. Alternatively, compositions may contain two or more anti-miRNA nucleic acids targeted to different regions, segments or sites of the same miRNA target. Two or more combined anti-miRNA compounds may be used together or sequentially.

In other embodiments, the nucleic acid is designed to target, at least in part, the seed region of the miRNA. Thus, in this embodiment, the target region includes at least a portion or the entire seed region of the miRNA. The term “seed region,” as used herein, refers to nucleotides at the 5′ end of the miRNA sequence that are typically common to an miRNA family. Examples of seed regions for certain miRNA's are set forth in Table 3 below (see underlined portion). In certain embodiments, the seed region includes 3, 4, 5, 6, 7, 8, 9, or 10 consecutive nucleotides within the miRNA sequence. Typically, the seed region of the miRNA is 6, 7, or 8 consecutive nucleotides within the miRNA sequence. For example, the seed region of the miRNA sequence may be nucleotides 1 through 7, 1 through 8, 2 through 7, 2 through 8, 1 through 9, 1 through 10, 2 through 9, 2 through 10, 3 through 10, or 4 through 12 from the 5′ end of the miRNA sequence. In some embodiments, the seed region of the miRNA sequence may advantageously be inclusively defined as nucleotides 1 through 7, 1 through 8, 2 through 7, or 2 through 8 from the 5′ end of the miRNA sequence.

The methods described herein (e.g. of increasing the concentration of a FKHR protein within the nucleus of a cell, decreasing cell viability in a cell, and treating cancer) include the use of a nucleic acid that is hybridizable to an RNA molecule, is antisense to an RNA molecule, is substantially complimentary to an RNA molecule, and/or has a sequence with at least 70% sequence identity to a 6 nucleobase (or nucleotide) sequence within one of SEQ ID NOs: 1-15 (also referred to herein as “anti-miRNA nucleic acid sequences”). See Table 2 for the complete sequences of SEQ ID NOs: 1-15. The RNA molecule in these methods is an miRNA selected from miRNA-17-5p, miRNA-25, miRNA-34b, miRNA-95, miRNA-99b, miRNA-150, miRNA-154, miRNA-204, miRNA-211, miRNA-328, miRNA-361, miRNA-422b, miRNA-449, miRNA-450-1, miRNA-508, and precursors thereof.

In certain embodiments, the nucleic acid comprises or consists of a sequence having at least 70% sequence identity to a 6, 7, 8, 9, 10, 11, 12, 13, 14, 15, 16, 17, 18, 19, 20, or 21 nucleobase sequence of or within one of SEQ ID NOs: 1 to 15. The “nucleobase sequence” refers to a consecutive nucleobases within the relevant SEQ ID NO. For example, the nucleic acid may comprise or consist of a sequence having at least 75%, at least 80%, at least 85%, at least 90%, at least 95%, at least 96%, at least 97%, at least 98%, at least 99%, or 100% sequence identity with any one of SEQ ID NOs: 1-15, or to a 6, 7, 8, 9, 10, 11, 12, 13, 14, 15, 16, 17, 18, 19, or 20 nucleobase sequence within any one of SEQ ID NOs: 1-15. In certain embodiments, the nucleic acid comprises or consists of a sequence having at least 75% or 80% sequence identity to one of SEQ ID NOs: 1-15. In other instances, the nucleic acid (the anti-miRNA nucleic acid sequence) has at least 85%, at least 90%, at least 95%, at least 96%, at least 97%, at least 98%, at least 99% or 100% sequence identity with any one of SEQ ID NOs: 1-15. In some embodiments, the nucleic acid comprises or consists of a sequence having 100% sequence identity with an anti-miRNA sequence (e.g. SEQ ID NO: 1, SEQ ID NO: 2, SEQ ID NO: 3, SEQ ID NO: 4, or SEQ ID NO: 5).

In some embodiments, the nucleic acid is at least 12 nucleobases in length. In other embodiments, the nucleic acid is at least 15 nucleobases in length. The nucleic acid may also be less than 22 nucleobases in length. Thus, in some embodiments, the nucleic acid is from 7 to 21 nucleobases in length. In other embodiments, the nucleic acid is from 8 to 21, 9 to 21, to 21, 11 to 21, 12 to 21, 13 to 21, 14 to 21, 15 to 21, 16 to 21, 17 to 21, or 18 to 21 nucleobases in length. In some instances, the nucleic acid is 7, 8, 9, 10, 11, 12, 13, 14, 15, 16, 17, 18, 19, 20, or 21 nucleobases in length.

Certain correlations between targeted RNA molecules and the respective nucleic acid anti-miRNA sequences are set forth in Table 2. Thus, as illustrated in Table 2, in some embodiments, the RNA molecule is an miRNA selected from miRNA-150, miRNA-204, miRNA-449, miRNA-450-1, miRNA-508, miRNA422b, miRNA-211, miRNA-328, miRNA-361, miRNA-25, miRNA-34b, miRNA-95, miRNA-99b, miRNA-17-5p, and miRNA-154 (and precursors thereof), and the nucleic acid comprises or consists of a sequence having at least 70% sequence identity to a nucleobase sequence of or within SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:1, SEQ ID NO:12, SEQ ID NO:13, SEQ ID NO: 14, and SEQ ID NO: 15, respectively. In other embodiments of the correlations set forth in Table 2, the nucleic acid comprises or consists of a sequence 7, 8, 9, 10, 11, 12, 13, 14, 15, 16, 17, 18, 19, 20, or 21 nucleobases, and having at least 70%, at least 75%, at least 80%, at least 85%, at least 90%, at least 95%, at least 96%, at least 97%, at least 98%, at least 99% or 100% sequence identity with the respective nucleic acid SEQ ID NOs: 1 to 15. In still other embodiments of the correlations set forth in Table 2, the RNA molecule is an miRNA selected from miRNA-150, miRNA-204, miRNA-449, miRNA-450-1, miRNA-508, or precursors thereof, and the respective nucleic acid has the appropriate number of nucleobases and the appropriate sequence identity as set forth in the previous sentence. The RNA molecule may also be miRNA-449, miRNA-450-1, and miRNA-508, or precursors thereof, and the respective nucleic acid (according to the correlations set forth in Table 2) has the appropriate number of nucleobases and the appropriate sequence identity as set forth above.

The nucleic acid may include a sequence that differs by no more than 8 nucleobases (or nucleotides) from any one of SEQ ID NOs: 1-15. In other embodiments, the nucleic acid may include a sequence that differs by no more than 5, 6, or 7 nucleobases (or nucleotides) from any one of SEQ ID NOs:1-15. In other embodiments, the nucleic acid may include a sequence that differs by no more than 1, 2, 3 or 4 nucleobases (or nucleotides) from any one of SEQ ID NOs:1-15 (e.g. SEQ ID NO: 1, SEQ ID NO: 2, SEQ ID NO: 3, SEQ ID NO: 4, or SEQ ID NO: 5).

As stated above, the nucleic acid may hybridize under stringent conditions to the RNA molecule. In some embodiment, the nucleic acid hybridizes under low stringency hybridization conditions to the RNA molecule. In other embodiments, the nucleic acid hybridizes under moderately stringent hybridization conditions to the RNA molecule. In other embodiments, the nucleic acid hybridizes under highly stringent hybridization conditions to the RNA molecule.

In some embodiments, the nucleic acid is substantially complementary to the miRNA or miRNA precursor. The nucleic acid may be at least 50%, at least 60%, at least 70%, at least 75%, at least 80%, or at least 85% sequence complementarity to a target region (e.g. seed region) within the miRNA or miRNA precursor. In other embodiments, the nucleic acid includes at least 90% sequence complementarity to a target region (e.g. seed region) within the miRNA or miRNA precursor. In other embodiments, the nucleic acid includes at least 95%, at least 96%, at least 97%, at least 98%, or at least 99% sequence complementarity to a target region (e.g. seed region) within the miRNA or miRNA precursor. For example, a nucleic acid in which 18 of 20 of its nucleobases are complementary to a target sequence (e.g. seed region) would represent 90 percent complementarity. Where a nucleic acid is substantially complementary to a miRNA or precursor, the remaining non-complementary nucleobases may be clustered or interspersed with complementary nucleobases and need not be contiguous to each other or to complementary nucleobases. Thus, a nucleic acid which is 22 nucleobases in length having 6 (six) non-complementary nucleobases which are flanked by two regions of complete complementarity with the target miRNA or miRNA precursor would have 72.7% overall complementarity with the miRNA or miRNA precursor. Percent complementarity of a nucleic acid with a region of an miRNA or miRNA precursor can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). In some embodiments, the nucleic acid is perfectly complementary to the miRNA or miRNA precursor.

Any appropriate method for introducing the nucleic acid into the cell may be employed. Examples of suitable methods include, for example, cell transfection methods such as chemical, biological or mechanical means. Recognized methods include electroporation, use of a virus vector, lipofection, gene guns, and microinjection.

The method may be practiced with any appropriate cell, such as a plant or animal cell. In some embodiments, the cell is a mammalian cell, such as a human cell. The cell may also be a cancer cell, such as a human breast cancer cell. Thus, in certain embodiments, the methods of introducing the nucleic acid into the cell are performed in vitro. Once into the cell, the nucleic acid increases the nuclear concentration of FKHR protein, or increases cell death (e.g. apoptosis), in situ.

### IV. METHODS OF TREATING CANCER

A variety of methods for treating cancer in a subject in need thereof are also provided. The methods include administering to the subject an effective amount of a nucleic acid that is hybridizable to an RNA molecule, is antisense to the RNA molecule, is substantially complimentary to the RNA molecule, and/or has a sequence with at least 70% sequence identity to a 6 nucleobase (or nucleotide) sequence within one of SEQ ID NOs: 1-15. In these methods, the RNA molecules that are targeted and the nucleic acids used to hybridize to the targeted RNA molecule are the same as those described above (i.e., in Section III describing the methods of modulating the concentration of FKHR in the nucleus of a cell and decreasing cell viability (e.g. increasing apoptosis and/or decreasing cellular proliferation) in a cell). Thus, the same sequence, length and other characteristics of the nucleic acids described in Section III apply equally to the methods for treating cancer.

In some embodiments, the effective amount is a therapeutically effective amount or a prophylactically effective amount. An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with cancer. A “therapeutically effective amount” is an amount sufficient to remedy a disease state (e.g. cancer) or symptoms, particularly a state or symptoms associated with the disease state, or otherwise prevent, hinder, retard or reverse the progression of the disease state or any other undesirable symptom associated with the disease in any way whatsoever. A “prophylactically effective amount” is an amount of a pharmaceutical composition that, when administered to a subject, will have the intended prophylactic effect, e.g., preventing or delaying the onset (or reoccurrence) of cancer, or reducing the likelihood of the onset (or reoccurrence) of cancer or cancer symptoms. The full prophylactic effect does not necessarily occur by administration of one dose, and may occur only after administration of a series of doses. Thus, a prophylactically effective amount may be administered in one or more administrations.

The RNA molecule is an miRNA selected from miRNA-17-5p, miRNA-25, miRNA-34b, miRNA-95, miRNA-99b, miRNA-150, miRNA-154, miRNA-204, miRNA-211, miRNA-328, miRNA-361, miRNA-422b, miRNA-449, miRNA-450-1, miRNA-508, and precursors thereof. See Table 3. The embodiments of the RNA molecule discussed in the section above are equally applicable to the methods of treating cancer. For example, in some embodiments, the RNA molecule is an miRNA selected from miRNA-150, miRNA-204, miRNA-449, miRNA-450-1, miRNA-508, and precursors thereof. The RNA molecule may also be miRNA-449, miRNA-450-1, and miRNA-508, or precursors thereof.

The nucleic acid is hybridizable to the RNA molecule, is antisense to the RNA molecule, is substantially complimentary to the RNA molecule, and/or has a sequence with at least 70% sequence identity to a 6 nucleobase (or nucleotide) sequence within one of SEQ ID NOs: 1-15″). See Table 2 for the complete sequences of SEQ ID NOs: 1-15. As with the RNA molecule embodiments, the embodiments of the nucleic acid discussed in Section III above are equally applicable to the methods of treating cancer. Thus, for example, the nucleic acid may comprise or consist of a sequence having at least 75%, at least 80% at least 85%, at least 90%, at least 95%, at least 96%, at least 97%, at least 98%, at least 99% or 100% sequence identity with an 6, 7, 8, 9, 10, 11, 12, 13, 14, 15, 16, 17, 18, 19, or 21 nucleobase sequence within any one of SEQ ID NOs: 1-15. Alternatively, the nucleic acid may comprise or consist of a sequence that differs by no more than 8 nucleobases (or nucleotides) from any one of SEQ ID NOs:1-15. In other embodiments, the nucleic acid may include a sequence that differs by no more than 5, 6, or 7 nucleobases (or nucleotides) from any one of SEQ ID NOs: 1-15. In other embodiments, the nucleic acid may include a sequence that differs by no more than 1, 2, 3 or 4 nucleobases (or nucleotides) from any one of SEQ ID NOs:1-15 (e.g. SEQ ID NO: 1, SEQ ID NO: 2, SEQ ID NO: 3, SEQ ID NO: 4, or SEQ ID NO: 5).

The nucleic acid can be administered by any suitable method that is effective in the treatment of the particular cancer or tumor type being treated. Thus, for instance, administration can be oral, rectal, topical, parenteral or intravenous administration or by injection into the tumor or cancer. The method of applying an effective amount also varies depending on the disorder or disease being treated. Parenteral treatment by intravenous, subcutaneous, or intramuscular application of the nucleic acid, formulated with an appropriate carrier, additional cancer inhibiting compound or compounds or diluent to facilitate application are suitable alternatives in administering the nucleic acid to a subject.

The nucleic acid may be combined with other active agents for use in combination therapies. For example, in some embodiments, the nucleic acid is combined with another anticancer agents, antiviral agent, and/or anticancer therapy. For example, other therapies or anticancer agents that may be used in combination with the nucleic acids described herein include surgery, radiotherapy (e.g. γ-radiation, neutron beam radiotherapy, electron beam radiotherapy, proton therapy, brachytherapy, and systemic radioactive isotopes), biologic response modifiers (e.g. interferons, interleukins, and tumor necrosis factor (TNF)), agents to attenuate any adverse effects (e.g. antiemetics), and other approved chemotherapeutic drugs, including, but not limited to, alkylating drugs (e.g. mechlorethamine, chlorambucil, Cyclophosphamide, Melphalan, Ifosfamide), antimetabolites (Methotrexate), purine antagonists and pyrimidine antagonists (e.g. 6-Mercapropurine, 5-Fluorouracil, Cytarabile, Gemeitabine), spindle poisons (e.g. Vinblastine, Vincristine, Vinorelbine, Paclitaxel), podophyllotoxins (e.g. Etoposide, Irinotecan, Topotecan), antibiotics (e.g. Doxorubicin, Bleomycin, Mixomycin), nitrosoureas (e.g. Carmustine, Lomustine), inorganic ions (e.g. Cisplatin, Carboplatin), enzymes (e.g. Asparaginase), Docetaxel, and hormones (e.g. Tamoxifen, Leuprolide, Flutamide, and Megestrol). A more comprehensive discussion of updated cancer therapies is provided in the list of FDA approved oncology drugs and The Merck Manual, Seventeenth Ed. 1999.

One skilled in the art will recognize that the efficacy of the nucleic acids can be ascertained through routine screening using known cancer cell lines both in vitro and in vivo. Cell lines are available from American Tissue Type Culture or other laboratories. The following examples are illustrative and not intended to be limit the scope of the methods described herein.

A. Identifying Subjects Amenable to Treatment

In some embodiments, subjects amenable treatment may be identified prior to the administration of a nucleic acid provided herein. Accordingly, methods provided herein may be used as a targeted therapy, wherein treatment is tailored to a subject based on the particular characteristics of the cancer cells in the subject. For instance, in murine hematopoietic system, it has been shown that the expression of miR-150 is restricted to spleenic B-cells, but not to pro-B cells and to naïve T-cells, but not to mature Th1 and Th2 cells. Subsequent studies revealed that B cell differentiation is attenuated when miR-150 is expressed prematurely. Consequently, high level of miR-150 expression has been shown in B cell lymphoma where lineage progression from Pro-B to Pre-B is impaired. Accordingly, methods provided herein may include identifying a subset of patients with B-cell malignancies with increased miR-150 expression leading to the pathological state and treating such patients with an oligonucleotide-based inhibitor that specifically targets an miR-150 sequence. An exemplary inhibitor includes the sequence set forth in SEQ ID NO: 1, and modified versions thereof, including those containing cholesterol-related moieties.

B. Measuring Response to Pharmaceutical Formulations

Tumor load is assessed prior to therapy by means of objective scans of the tumor such as with x-ray radiographs, computerized tomography (CAT scans), nuclear magnetic resonance (NMR) scans or direct physical palpation of the tumor mass. Alternatively, the tumor may secrete a marker substance such as alphafetoprotein from colon cancer, CA 125 antigen from ovarian cancer, or serum myeloma “M” protein from multiple myeloma. The levels of these secreted products then allow for an estimate of tumor burden to be calculated. These direct and indirect measures of the tumor load are done pretherapy, and are then repeated at intervals following the administration of the drug in order to gauge whether or not an objective response has been obtained. An objective response in cancer therapy generally indicates >50% shrinkage of the measurable tumor disease (a partial response), or complete disappearance of all measurable disease (a complete response). Typically these responses must be maintained for a certain time period, usually one month, to be classified as a true partial or complete response. In addition, there may be stabilization of the rapid growth of a tumor or there may be tumor shrinkage that is <50%, termed a minor response or stable disease. In general, increased survival is associated with obtaining a complete response to therapy and in some cases, a partial response if maintained for prolonged periods can also contribute to enhanced survival in the patient. Patients receiving chemotherapy are also typically “staged” as to the extent of their disease before and following chemotherapy are then restaged to see if this disease extent has changed. In some situations the tumor may shrink sufficiently and if no metastases are present, then surgical excision may be possible after chemotherapy treatment where it was not possible beforehand due to the widespread disease. In this case the chemotherapy treatment with the novel pharmaceutical compositions is being used as an adjuvant to potentially curative surgery. In addition, patients may have individual lesions in the spine or elsewhere that produce symptomatic problems such as pain and these may need to have local radiotherapy applied. This may be done in addition to the continued use of the systemic pharmaceutical compositions.

C. Assessing Toxicity and Setting Dosing Regimens

Patients are assessed for toxicity with each course of nucleic acid administration typically looking at effects on liver function enzymes and renal function enzymes such as creatinine clearance or BUN as well as effects on the bone marrow, typically a suppression of granulocytes important for fighting infection and/or a suppression of platelets important for hemostasis or stopping blood flow. For such assessments, normal blood counts may be reached between 1-3 weeks after therapy. Recovery then ensues over the next 1-2 weeks. Based on the recovery of normal white blood counts, treatments may then be resumed.

In general, complete and partial responses are associated with at least a 1-2 log reduction in the number of tumor cells (a 90-99% effective therapy). Patients with advanced cancer will typically have >10⁹ tumor cells at diagnosis, multiple treatments will be required in order to reduce tumor burden to a very low state and potentially obtain a cure of the disease.

D. Clinical Management of Patients

At the end of a treatment cycle with a nucleic acid which could comprise several weeks of continuous drug dosing, patients can be evaluated for response to therapy (complete and partial remissions), toxicity measured by blood work and general well-being classified performance status or quality of life analysis. The latter includes the general activity level of the patient and their ability to do normal daily functions. It has been found to be a strong predictor of response and some anticancer drugs may actually improve performance status and a general sense of well-being without causing a significant tumor shrinkage. Thus, for some cancers that are not curable, the pharmaceutical formulations may similarly provide a significant benefit, well-being performance status, etc. without affecting true complete or partial remission of the disease.

In hematologic disorders such as multiple myeloma, lymphoma and leukemia, responses are not assessed via the measurement of tumor diameter since these diseases are widely metastatic throughout the lymphatic and hematogenous areas of the body. Thus, responses to these diffusely disseminated diseases are usually measured in terms of bone marrow biopsy results wherein the number of abnormal tumor cell blasts are quantitated and complete responses are indicated by the lack of detection (e.g. microscopic detection) of any tumor cells in a bone marrow biopsy specimen. With the B-cell neoplasm multiple myeloma a serum marker, the M protein, can be measured by electrophoresis and if substantially decreased this is evidence of the response of the primary tumor. Again, in multiple myeloma, bone marrow biopsies can be used to quantitate the number of abnormal tumor plasma cells present in the specimen. For these diseases generally higher dose therapy is typically used to affect responses in the bone marrow and/or lymphatic compartments.

The projected clinical uses for the novel pharmaceutical formulations are as treatments for: lung cancer, breast cancer, malignant melanoma, AIDS-related lymphoma, multidrug-resistant (MDR) tumors (Myeloma, Leukemia Breast and Colon Carcinoma), prostate cancer, multiple myeloma, a β-lymphocyte plasmacytoma, advanced stage ovarian epithelial cell cancer, metastatic melanoma, leukemias of lymphoid and nonlymphoid origin, metastatic colon cancer, breast cancers and metastatic lung cancers, and neoplasms of the endocrine and exocrine pancreas.

E. Cancer Assays

A number of biological assays are available to evaluate and to optimize the choice of nucleic acids for optimal antitumor activity. These assays can be roughly split into two groups those involving in vitro exposure of nucleic acids to tumor cells and in vivo antitumor assays in rodent models and rarely, in larger animals.

Cytotoxic assays in vitro for nucleic acids generally involve the use of established tumor cell lines both of animal and, especially of human origin. These cell lines can be obtained from commercial sources such as the American Type Tissue Culture Laboratory in Bethesda, Md. and from tumor banks at research institutions. Exposures to nucleic acids may be carried out under simulated physiological conditions of temperature, oxygen and nutrient availability in the laboratory. The endpoints for these in vitro assays can involve: 1) colony formation; 2) a simple quantitation of cell division over time; 3) the uptake of so called “vital” dyes which are excluded from cells with an intact cytoplasmic membrane; 4) the incorporation of radiolabeled nutrients into a proliferating (viable) cell. Colony forming assays have been used both with established cell lines, as well as fresh tumor biopsies surgically removed from patients with cancer. In this type of assay, cells are typically grown in petri dishes on soft agar, and the number of colonies or groups of cells (>60μ in size) are counted either visually, or with an automated image analysis system. A comparison is then made to the untreated control cells allowed to develop colonies under identical conditions. Because colony formation is one of the hallmarks of the cancer phenotype, only malignant cells will form colonies without adherence to a solid matrix. This can therefore be used as a screening procedure and assay for effectiveness for nucleic acids, and there are a number of publications which show that results obtained in colony forming assays correlates with clinical trial findings with the same drugs.

The enumeration of the total number of cells is one approach to in vitro testing with either cell lines or fresh tumor biopsies. In this assay, clumps of cells are typically disaggregated into single units which can then be counted either manually on a microscopic grid or using an automated flow system such as either flow cytometry or a Coulter® counter. Control (untreated) cell growth rates are then compared to the treated (with a nucleic acid) cell growth rates. Vital dye staining is another one of the older hallmarks of antitumor assays. In this type of approach cells, either untreated or treated with a cancer drug, are subsequently exposed to a dye such as methylene blue, which is normally excluded from intact (viable) cells. The number of cells taking up the dye (dead or dying) are the numerator with a denominator being the number of cells which exclude the dye.

In addition to vital dye staining, viability can be assessed using the incorporation of radiolabeled nutrients and/or nucleotides. In tumor cell assays, a typical experiment involves the incorporation of either (³H) tritium or ¹⁴C-labeled nucleotides such as thymidine. Control (untreated) cells are shown to take up a substantial amount of this normal DNA building block per unit time, and the rate of incorporation is compared to that in the drug treated cells. This is a rapid and easily quantitatable assay that has the additional advantage of working well for cells that may not form large (countable) colonies. Drawbacks include the use of radioisotopes which present handling and disposal concerns.

There are large banks of human and rodent tumor cell lines that are available for these types of assays. The current test system used by the National Cancer Institute uses a bank of over 60 established sensitive and multidrug-resistant human cells lines of a variety of cell subtypes. This typically involves 5-6 established and well-characterized human tumor cells of a particular subtype, such as non-small cell or small cell lung cancer, for testing new agents. Using a graphic analysis system called Compare®, the overall sensitivity in terms of dye uptake (either sulforhodamine B or MTT tetrazolium dye) are utilized. The specific goal of this approach is to identify nucleic acids that are uniquely active in a single histologic subtype of human cancer. In addition, there are a few sublines of human cancer that demonstrate resistance to multiple agents and are known to, in some cases, express the multidrug resistance pump, p-glycoprotein. The endpoint for certain assays is the incorporation of a protein dye called sulforhodamine B (for adherent tumor cells) and the reduction of a tetrazolium (blue) dye in active mitochondrial enzymes (for non-adherent, freely-floating types of cells). This latter method is particularly useful for hematologic cancers including myelomas, leukemias and lymphomas.

Generally, once a nucleic acid has demonstrated some degree of activity in vitro at inhibiting tumor cell growth, such as colony formation or dye uptake, antitumor efficacy experiments are performed in vivo. Rodent systems can be used for initial assays of antitumor activity since tumor growth rates and survival endpoints are well-defined, and since these animals generally reflect the same types of toxicity and drug metabolism patterns as in humans. For this work, syngeneic (same gene line) tumors are typically harvested from donor animals, disaggregated, counted and then injected back into syngeneic (same strain) host mice. Anticancer nucleic acids are typically then injected at some later time point(s), either by intraperitoneal or intravenous administration, or administered by the oral routes, Tumor growth rates and/or survival are determined and compared to untreated controls. In these assays, growth rates are typically measured for tumors growing in the front flank of the animal, wherein perpendicular diameters of tumor width are translated into an estimate of total tumor mass or volume. The time to reach a predetermined mass is then compared to the time required for equal tumor growth in the untreated control animals. In some embodiments, significant findings generally involve a >25% increase in the time to reach the predetermined mass in the treated animals compared to the controls. In other embodiments, significant findings involve a >42% increase in the time to reach the predetermined mass in the treated animals compared to the controls. The significant findings are termed tumor growth inhibition. For non-localized tumors such as leukemia, survival can be used as an endpoint and a comparison is made between the treated animals and the untreated or solvent treated controls. In general, a significant increase in life span for a positive new agent is again >20-42% longer life span due to the treatment. Early deaths, those occurring before any of the untreated controls, generally indicate toxicity for a new compound.

For all these assays, the anticancer nucleic acids are generally tested at doses very near the lethal dose and 10% (LD₁₀) and/or at the determined maximally-tolerated dose, that dose which produces significant toxicity, but no lethality in the same strain of animals and using the same route of administration and schedule of dosing. Similar studies can also be performed in rat tumor models.

More recently, human tumors have been successfully transplanted in a variety of immunologically deficient mouse models. A mouse called the nu/nu or “nude” mouse can be used to develop in vivo assays of human tumor growth. In nude mice, which are typically hairless and lack a functional thymus gland, human tumors (millions of cells) are typically injected in the flank and tumor growth occurs slowly thereafter. This visible development of a palpable tumor mass is called a “take”. Anticancer drugs such as the nucleic acids disclosed herein are then injected by some route (IV, IM, SQ, PO) distal to the tumor implant site, and growth rates are calculated by perpendicular measures of the widest tumor widths as described earlier. A number of human tumors are known to successfully “take” in the nude mouse model. An alternative mouse model for this work involves mice with a severe combined immunodeficiency disease (SCID), in which there is a defect in maturation of lymphocytes. Because of this, SCID mice do not produce functional B- and T-lymphocytes. However, these animals do have normal cytotoxic T-killer cell activity. Nonetheless, SCID mice will “take” a large number of human tumors. Tumor measurements and drug dosing are generally performed as above. Again, positive compounds in the SCID mouse model are those that inhibit tumor growth rate by >20-42% compared to the untreated control.

Testing for drug resistance can involve any of the in vitro and in vivo models. In these tests, a cell subline is developed for resistance to a particular agent generally by serial exposure to increasing concentrations of the anticancer nucleic acids either in vitro or rarely in vivo. Once a high degree of resistance is demonstrated (generally >4- to 5-fold) to a particular agent, the cell line is further studied for mechanisms of resistance such as the expression of multidrug resistance membrane pumps such as p-glycoprotein or others. These resistant cell lines can then be tested for cross-resistance with classic anticancer agents to develop a response pattern for a particular cell line. Using this cell line one can then evaluate a new agent for its potential to be active in the resistant cells. This has allowed for the demonstration of both mechanisms of drug resistance, as well as the identification of agents which might have utility in human cancers that have become resistant to existing chemotherapy agents. More recently, the use of resistant human tumor cells has been extended to the SCID mouse model with the development of an in vivo model of multidrug-resistant human multiple myeloma.

All of these test systems are generally combined in a serial order, moving from in vitro to in vivo, to characterize the antitumor activity of an anticancer nucleic acid. In general, one wishes to find out what tumor types are particularly sensitive to a nucleic acid and conversely what tumor types are intrinsically resistant to a nucleic acid in vitro. Using this information, experiments are then planned in rodent models to evaluate whether or not the nucleic acids that have shown activity in vitro will be tolerated and active in animals. The initial experiments in animals generally involve toxicity testing to determine a tolerable dose schedule and then using that dose schedule, to evaluate antitumor efficacy as described above. Active nucleic acids from these two types of assays may then be tested in human tumors growing in SCID or nude mice and if activity is confirmed, these nucleic acids then become candidates for potential clinical drug development.

### V. NUCLEIC ACIDS AND GENERAL NUCLEIC ACID SYNTHESES

A. Types of Nucleic Acids

The nucleic acid may be modified to increase stability of the nucleic acids toward nucleases, to increase hybridization stability, or to increase inhibition of miRNA or miRNA precursor function. In some embodiments, the nucleic acid includes modifications to the standard phosphodiester linkages found in natural or unmodified nucleic acids. Modified nucleic acid backbones (internucleotide linkages) containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. The preparation of the above phosphorus-containing linkages is discussed in greater detail below and, for example, in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference. In some embodiments, the nucleic acid includes one or more modified internucleotide or internucleoside linkages selected from phosphoroamidate, phosphorothiate, phosphorodithioate, boranophosphate, alkylphosphonate, and methylinemethylimino. For further description of methylinemethylimino internucleoside linkages, see U.S. Pat. Nos. 5,378,825, 5,386,023, 5,489,677, 5,602,240, and 5,610,289, each of which is herein incorporated by reference. Appropriate mixed backbone nucleic acid linkages, with standard phosphodiester linkages or with one or more different modified internucleotide or internucleoside linkages, are useful in the methods described herein.

The nucleic acid may also include a modified nucleic acid unit selected from a locked nucleic acid unit, 2′-O-methyl ribonucleic acid unit, 2′O-methoxy-ethyl ribonucleic acid unit, 2′alkyl ribonucleic acid unit, 2′amine ribonucleic acid unit, peptide nucleic acid unit, 2′fluoro-ribo nucleic acid unit, morpholino nucleic acid unit, cyclohexane nucleic acid unit, or a tricyclonucleic acid unit. For further information regarding modified nucleic acid units, see U.S. App. No. 2005/0182005, which is herein incorporated by reference. In some embodiments, the nucleic acid is a locked nucleic acid, a 2′-O-methyl ribonucleic acid, or a mixed nucleic acid-locked nucleic acid.

In some embodiments, a nucleic acid is provided having at least 30%, 40%, 50%, 60%, 70%, 80%, 85%, or 90% locked nucleic acid units. In other embodiments, the nucleic acid includes at least 80%, 85%, or 90% locked nucleic acid units. The nucleic acid is hybridizable to an RNA molecule, is antisense to an RNA molecule, is substantially complimentary to an RNA molecule, and/or has a sequence with at least 70% sequence identity to a 6 nucleobase (or nucleotide) sequence within one of SEQ ID NOs: 1-15. The RNA molecule is an miRNA selected from miRNA-17-5p, miRNA-25, miRNA-34b, miRNA-95, miRNA-99b, miRNA-150, miRNA-154, miRNA-204, miRNA-211, miRNA-328, miRNA-361, miRNA-422b, miRNA-449, miRNA-450-1, miRNA-508, and precursors thereof. Thus, in some embodiments, the nucleic acid includes at least 91%, 92%, 93%, 94%, 95%, 96%, 97%, 98%, 99%, or 100% locked nucleic acid units. Where a nucleic acid includes a defined percentage of locked nucleic acid units, the percentage is the number of locked nucleic acid units divided by the total number of nucleic acid units multiplied by 100%. In some embodiments, all of the nucleic acid units within the nucleic acid are locked nucleic acid units with the exception of 1, 2, 3, 4 or 5 nucleic acid units (e.g., nucleotides). In some embodiments, the internucleotide linkages are phosphodiester linkages or phosphorothioate linkages. Any nucleic acid units that are not locked nucleic acid units may be selected from ribonucleic acid units, deoxyribonucleic acid units, and 2′-O-methyl nucleic acid units. The nucleic acid may be any appropriate length as described in Section III below (e.g. 7, 8, 9, 10, 11, 12, 13, 14, 15, 16, 17, 18, 19, 20, or 21 nucleobases in length). In some embodiments, the nucleic acid has at least 75%, at least 80%, at least 85%, at least 90%, at least 95%, at least 96%, at least 97%, at least 98%, at least 99% or 100% sequence identity with any one of SEQ ID NOs: 1-15, or to a 6, 7, 8, 9, 10, 11, 12, 13, 14, 15, 16, 17, 18, 19, or nucleobase sequence within any one of SEQ ID NOs: 1-15.

The nucleic acids can have one or more moieties bound or conjugated, which facilitates the active or passive transport, localization, or compartmentalization of the nucleic acid. Cellular localization includes, but is not limited to, localization to within the nucleus, the nucleolus, or the cytoplasm. Compartmentalization includes, but is not limited to, any directed movement of the nucleic acids compounds to a cellular compartment including the nucleus, nucleolus, mitochondrion, or imbedding into a cellular membrane.

One substitution that can be appended to the nucleic acids involves the linkage of one or more moieties or conjugates which enhance the activity, cellular distribution or cellular uptake of the resulting nucleic acids. In one embodiment such modified nucleic acids are prepared by covalently attaching conjugate groups to functional groups such as hydroxyl or amino groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, carbohydrates, lipids, phospholipids, biotin, phenazine, folate, phenanthridine, anthraquinone, acridine, fluoresceins, rhodamines, coumarins, and dyes. Groups that enhance the pharmacodynamic properties include groups that improve oligomer uptake, enhance oligomer resistance to degradation, and/or strengthen hybridization with RNA. Groups that enhance the pharmacokinetic properties include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glyc-ero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). For example, nucleic acids described herein may optionally include one or more cholesterol moieties suitably linked to the nucleic acid such that the intracellular resident time of the nucleic acid is increased. In some aspects, a mono-cholesterol-linked to a nucleic acid increases the intracellular resident time of the nucleic acid. In other aspects, a plurality of cholesterol moieties may be linked to a nucleic acid to increase the intracellular resident time of the nucleic acid in a cell.

The nucleic acids may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Nucleic acid-drug conjugates and their preparation are described in U.S. patent application Ser. No. 09/334,130 (filed Jun. 15, 1999) which is incorporated herein by reference in its entirety.

Representative U.S. patents that teach the preparation of such nucleic acid conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

Nucleic acids can also be modified to have one or more stabilizing groups that are generally attached to one or both termini of nucleic acids to enhance properties such as for example nuclease stability. Included in stabilizing groups are cap structures. By “cap structure or terminal cap moiety” is meant chemical modifications, which have been incorporated at either terminus of nucleic acids (see for example Wincott et al., WO 97/26270, incorporated by reference herein). These terminal modifications protect the nucleic acids having terminal nucleic acid molecules from exonuclease degradation, and can help in delivery and/or localization within a cell. The cap can be present at the 5′-terminus (5′-cap) or at the 3′-terminus (3′-cap) or can be present on both termini. For double-stranded nucleic acids, the cap may be present at either or both termini of either strand. In non-limiting examples, the 5′-cap includes inverted abasic residue (moiety), 4′,5′-methylene nucleotide; 1-(beta-D-erythrofuranosyl)nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (see Wincott et al., International PCT publication No. WO 97/26270, incorporated by reference herein).

Useful 3′-cap structures include, for example 4′,5′-methylene nucleotide; 1-(beta-D-erythrofuranos-yl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925; incorporated by reference herein). Further 3′ and 5′-stabilizing groups that can be used to cap one or both ends of a nucleic acid to impart nuclease stability include those disclosed in WO 03/004602 published on Jan. 16, 2003.

B. General Nucleic Acid Syntheses

Oligomerization of modified and unmodified nucleosides is performed according to literature procedures for DNA like compounds (see, e.g., Protocols for Oligonucleotides and Analogs, Ed. Agrawal (1993), Humana Press) and/or RNA like compounds (see, e.g., Scaringe, Methods (2001), 23, 206-217; Gait et al., Applications of Chemically synthesized RNA in RNA:Protein Interactions, Ed. Smith (1998), 1-36; Gallo et al., Tetrahedron (2001), 57, 5707-5713) synthesis as appropriate. In addition, some examples of protocols for the synthesis of nucleic acids are illustrated below.

RNA can be synthesized by methods disclosed herein or purchased from various RNA synthesis companies (e.g. Dharmacon Research Inc., (Lafayette, Colo.)).

Regardless of the particular protocol used, the nucleic acids used herein may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

The following compounds, including amidites and their intermediates can be prepared as described in U.S. Pat. No. 6,426,220 and published PCT WO 02/36743; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5′-O-Dimethoxytrityl-2′-deoxy-N4-benzoyl-5-methylcyt-idine penultimate intermediate for 5-methyl dC amidite, (5′-O-(4,4′-Dimethoxytriphenylmethyl)-2′-deoxy-N.sup.4-benzoyl-5-methylcy-tidin-3′-O-yl)-2-cyanoethyl-N,N-diisopropylphosphoramidite (5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, (5′-O-(4,4′-Dimethoxytriphenylmethyl)-2′-O-(2-methoxyethyl)-5-methyluridi-n-3′-O-yl)-2-cyanoethyl-N,N-diisopropylphosphoramidite (MOE T amidite), 5′-O-Dimethoxytrityl-2′-O-(2-methoxyethyl)-5-methylcytidine intermediate, 5′-O-dimethoxytrityl-2′-O-(2-methoxyethyl)-N.sup.4-benzoyl-5-methyl-cytid-ine penultimate intermediate, (5′-O-(4,4′-Dimethoxytriphenylmethyl)-2′-O-(-2-methoxyethyl)-N.sup.4-benzoyl-5-methylcytidin-3′-O-yl)-2-cyanoethyl-N,N-diisopropylphosphoramidite (MOE 5-Me-C amidite), (5′-O-(4,4′-Dimethoxytrip-henylmethyl)-2′-O-(2-methoxyethyl)-N.sup.6-benzoyladenosin-3′-O-yl)-2-cyan-oethyl-N,N-diisopropylphosphoramidite (MOE A amdite), (5′-O-(4,4′-Dimethoxytriphenylmethyl)-2′-O-(2-methoxyethyl)-N.sup.4-isobu-tyrylguanosin-3′-O-yl)-2-cyanoethyl-N,N-diisopropylphosphoramidite (MOE G amidite), 2′-O-(Aminooxyethyl)nucleoside amidites and 2′-O-(dimethylaminooxyethyl)nucleoside amidites, 2′-(Dimethylaminooxyetho-xy)nucleoside amidites, 5′-O-tert-Butyldiphenylsilyl-O.sup.2-2′-anhydro-5-methyluridine, 5′-O-tert-Butyldiphenylsilyl-2′-O-(2-hydroxyethyl)-5-methyl-uridine, 2′-O-((2-phthalimidoxy)ethyl)-5′-t-butyldiphenylsilyl-5-methyluri-dine, 5′-O-tert-butyldiphenylsilyl-2′-O-((2-formadoximinooxy)ethyl)-5-meth-yluridine, 5′-O-tert-Butyldiphenylsilyl-2′-O—(N,N dimethylaminooxyethyl)-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-((2-cyanoe-thyl)-N,N-diisopropylphosphoramidite), 2′-(Aminooxyethoxy)nucleoside amidites, N2-isobutyryl-6-O-diphenylcarbamoyl-2′-O-(2-ethylacetyl)-5′-O-(-4,4′-dimethoxytrityl)guanosine-3′-((2-cyanoethyl)-N,N-diisopropylphosphora-midite), 2′-dimethylaminoethoxyethoxy(2′-DMAEOE)nucleoside amidites, 2′-O-(2(2-N,N-dimethylaminoethoxy)ethyl)-5-methyl uridine, 5′-O-dimethoxytrityl-2′-O-(2(2-N,N-dimethylaminoethoxy)-ethyl))-5-methyl uridine and 5′-O-Dimethoxytrityl-2′-O-(2(2-N,N-dimethylaminoethoxy)-ethyl-))-5-methyl uridine-3′-O-(cyanoethyl-N,N-diisopropyl)phosphoramidite.

Unsubstituted and substituted phosphodiester (P═O) nucleic acids can be synthesized on an automated nucleic acid synthesizer (Applied Biosystems model 394) using standard phosphoramidite chemistry with oxidation by iodine. Generally, nucleic acids can be cleaved from solid support (e.g. a controlled pore glass column) and deblocked in concentrated ammonium hydroxide, then recovered by precipitation using NH₄OAc with ethanol. Synthesized nucleic acids may be analyzed by electrospray mass spectroscopy (molecular weight determination) and by capillary gel electrophoresis.

Phosphorothioates (P═S) can be synthesized similar to phosphodiester nucleic acids with the following exceptions: thiation is effected by utilizing a 10% w/v solution of 3,H-1,2-benzodithiole-3-one 1,1-dioxide in acetonitrile for the oxidation of the phosphite linkages. The thiation reaction step time is increased to 180 sec and preceded by the normal capping step. After cleavage from the solid support and deblocking in concentrated ammonium hydroxide at the appropriate temperature, the nucleic acids may be recovered by precipitating with ethanol from a 1 M NH₄OAc solution. Phosphinate nucleic acids can be prepared as described in U.S. Pat. No. 5,508,270, herein incorporated by reference.

Alkyl phosphonate nucleic acids can be prepared as described in U.S. Pat. No. 4,469,863, herein incorporated by reference.

3′-Deoxy-3′-methylene phosphonate nucleic acids can be prepared as described in U.S. Pat. No. 5,610,289 or 5,625,050, herein incorporated by reference.

Phosphoramidite nucleic acids can be prepared as described in U.S. Pat. No. 5,256,775 or U.S. Pat. No. 5,366,878, herein incorporated by reference.

Alkylphosphonothioate nucleic acids can be prepared as described in published PCT applications PCT/US94/00902 and PCT/US93/06976 (published as WO 94/17093 and WO 94/02499, respectively), herein incorporated by reference.

3′-Deoxy-3′-amino phosphoramidate nucleic acids can be prepared as described in U.S. Pat. No. 5,476,925, herein incorporated by reference.

Phosphotriester nucleic acids can be prepared as described in U.S. Pat. No. 5,023,243, herein incorporated by reference.

Boranophosphate nucleic acids can be prepared as described in U.S. Pat. Nos. 5,130,302 and 5,177,198, both herein incorporated by reference.

Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone nucleic acids having, for instance, alternating MMI and P═O or P═S linkages can be prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides can be prepared as described in U.S. Pat. Nos. 5,264,562 and 5,264,564, herein incorporated by reference.

Ethylene oxide linked oligonucleosides can be prepared as described in U.S. Pat. No. 5,223,618, herein incorporated by reference.

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers can be used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that can be differentially removed and can be differentially chemically labile, RNA nucleic acids were synthesized.

RNA nucleic acids can be synthesized in a stepwise fashion. In this approach, each nucleotide is added sequentially (3′- to 5′-direction) to a solid support-bound nucleic acid. The first nucleoside at the 3′-end of the chain is covalently attached to a solid support. The nucleotide precursor, a ribonucleoside phosphoramidite, and activator can be added, coupling the second base onto the 5′-end of the first nucleoside. The support is washed and any unreacted 5′-hydroxyl groups can be capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide. Following synthesis, the methyl protecting groups on the phosphates can be cleaved utilizing 1 M disodium-2-carbamoyl-2-cyanoethyl-ene-1,1-dithiolate trihydrate (S₂Na₂) in DMF. The deprotection solution is washed from the solid support-bound nucleic acid using water. The support is then treated with 40% methylamine in water. This releases the RNA nucleic acids into solution, deprotects the exocyclic amines, and modifies the 2′-groups. The nucleic acids can be analyzed by anion exchange HPLC at this stage.

The 2′-orthoester groups can be the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and nucleic acid synthesis. However, after nucleic acid synthesis the nucleic acid is treated with methylamine which not only cleaves the nucleic acid from the solid support but also removes the acetyl groups from the orthoesters. The resulting 2-ethyl-hydroxyl substituents on the orthoester can be less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with nucleic acid synthesis and yet, when subsequently modified, permits deprotection to be carried out under relatively mild aqueous conditions compatible with the final RNA nucleic acid product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand,. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

Nucleic acids incorporating at least one 2′-O-protected nucleoside may also be prepared. After incorporation and appropriate deprotection the 2′-O-protected nucleoside will be converted to a ribonucleoside at the position of incorporation. The number and position of the 2-ribonucleoside units in the final nucleic acid can vary from one at any site or the strategy can be used to prepare up to a full 2′-OH modified nucleic acid. All 2′-O-protecting groups amenable to the synthesis of nucleic acids are included herein.

In general a protected nucleoside is attached to a solid support by for example a succinate linker. Then the nucleic acid is elongated by repeated cycles of deprotecting the 5′-terminal hydroxyl group, coupling of a further nucleoside unit, capping and oxidation (alternatively sulfurization). In a more frequently used method of synthesis the completed nucleic acid is cleaved from the solid support with the removal of phosphate protecting groups and exocyclic amino protecting groups by treatment with an ammonia solution. Then a further deprotection step is normally required for the more specialized protecting groups used for the protection of 2′-hydroxyl groups which will give the fully deprotected nucleic acid.

An effective 2′-O-protecting group is typically capable of selectively being introduced at the 2′-O-position and can be removed easily after synthesis without the formation of unwanted side products. The protecting group is usually inert to the normal deprotecting, coupling, and capping steps required for oligoribonucleotide synthesis. Examples of protecting groups include tetrahydropyran-1-yl, 4-methoxytetrahydropyran-4-yl, piperidine derivatives (e.g. Fpmp) (Reese et al., Tetrahedron Lett., 1986, (27), 2291), standard 5′-DMT (dimethoxytrityl) group, t-butyldimethylsilyl group (Ogilvie et al., Tetrahedron Lett., 1974, 2861; Hakimelahi et al., Tetrahedron Lett., 1981, (22), 2543; and Jones et al., J. Chem. Soc. Perkin I., 2762), fluoride labile and photolabile protecting groups (e.g. the 2-(nitrobenzyl)oxy)methyl (nbm) protecting group (Schwartz et al., Bioorg. Med. Chem. Lett., 1992, (2), 1019)), formaldehyde acetal-derived, 2′-O-protecting groups, 2′-O-alkylated nucleoside phosphoramidites including 2′-O-((triisopropylsilyl)oxy)methyl(2′-O—CH₂—O—Si(iPr)₃TOM), fluoride labile 5′-O-protecting group (non-acid labile) and an acid labile 2′-O-protecting group (Scaringe, Stephen A., Methods, 2001, (23) 206-217). A particularly useful protection scheme is a 5′-O-silyl ether-2′-ACE (5′-O-bis(trimethylsiloxy)cyclododecyloxysilyl ether (DOD)-2′-O-bis(2-acetoxyethoxy)methyl (ACE). This approach uses a modified phosphoramidite synthesis approach in that some different reagents are required that are not routinely used for RNA/DNA synthesis.

RNA synthesis strategies used commercially include 5′-O-DMT-2′-O-t-butyldimethylsilyl (TBDMS), 5′-O-DMT-2′-O-(1(2-fluorophenyl)-4-methoxypiperidin-4-yl) (FPMP), 2′-O-((triisopropylsilyl)oxy)methyl(2′-O—CH₂—O—Si(iPr)₃ (TOM), and the 5′-O-silyl ether-2′-ACE (5′-O-bis(trimethylsiloxy)cyclod-odecyloxysilyl ether (DOD)-2′-O-bis(2-acetoxyethoxy)methyl (ACE). A current list of some of the major companies currently offering RNA products include Pierce Nucleic Acid Technologies, Dharmacon Research Inc., Ameri Biotechnologies Inc., and Integrated DNA Technologies, Inc

Nucleic acids may also be synthesized via solid phase P(III) phosphoramidite chemistry on an automated synthesizer capable of assembling 96 sequences simultaneously in a 96-well format. Phosphodiester internucleotide linkages can be afforded by oxidation with aqueous iodine. Phosphorothioate internucleotide linkages can be generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites can be purchased from commercial vendors (e.g. PE-Applied Biosystems, Foster City, Calif., or Pharmacia, Piscataway, N.J.). Non-standard nucleosides can be synthesized as per standard or patented methods. They can be utilized as base protected beta-cyanoethyldiisopropyl phosphoramidites.

Modified nucleic acid backbones (internucleoside linkages) that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, and each of which is herein incorporated by reference.

Another group of nucleic acids amenable to the methods provided herein include nucleic acid mimetics. The term mimetic as it is applied to nucleic acids is intended to include nucleic acids wherein only the furanose ring or both the furanose ring and the internucleotide linkage can be replaced with novel groups, replacement of only the furanose ring is also referred to in the art as being a sugar surrogate. The heterocyclic base moiety or a modified heterocyclic base moiety is maintained for hybridization with an appropriate target nucleic acid. One such nucleic acid mimetic compound that has been shown to have excellent hybridization properties, is referred to as a peptide nucleic acid (PNA). In PNA nucleic acids, the sugar-backbone of an nucleic acid is replaced with an amide containing backbone, in particular an aminoethylglycine backbone. The nucleobases can be retained and bound directly or indirectly to aza nitrogen atoms of the amide portion of the backbone. Representative U.S. patents that teach the preparation of PNA nucleic acids include, but are not limited to, U.S. Pat. Nos. 5,539,082; 5,714,331; and 5,719,262, each of which is herein incorporated by reference. A discussion of PNA nucleic acids can be found in Nielsen et al., Science, 1991, 254, 1497-1500.

Other nucleic acid mimetics that can be used include nucleosides having sugar moieties that are bicyclic thereby locking the sugar conformational geometry. One example of such a nucleotide is a bicyclic sugar moiety having a 4′-CH₂—O-2′ bridge. The 2′-O— has been linked via a methylene group to the 4′ carbon (see U.S. patent application Publication No. application 2003/0087230). The xylo analog has also been prepared (see U.S. patent application Publication No. 2003/0082807). The bridge for a locked nucleic acid (LNA) may be 4′-(—CH₂—)₂—O-2′ wherein n is 1 or 2 (Kaneko et al., U.S. patent application Publication No. US 2002/0147332, Singh et al., Chem. Commun., 1998, 4, 455-456, also see U.S. Pat. Nos. 6,268,490 and 6,670,461 and U.S. patent application Publication No. US 2003/0207841). However the term locked nucleic acids can also be used in a more general sense to describe any bicyclic sugar moiety that has a locked conformation.

Potent and nontoxic antisense nucleic acids containing LNAs have been described (Wahlestedt et al., Proc. Natl. Acad. Sci. U.S.A., 2000, 97, 5633-5638.). The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226. The first analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., PCT International Application WO 98-DK393 19980914). Furthermore, synthesis of 2′-amino-LNA, a novel conformationally restricted high-affinity nucleic acid analog with a handle has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported. Also see U.S. Patent Application No. 20050261218.

### VI. ASSAYS

Nucleic acids may be easily tested for their ability to hybridize to an RNA molecule, increase FKHR concentration in the nucleus, and/or decrease cell viability (e.g. increase cell apoptosis) using assays well-known in the art and described herein.

For example, in some assays to test whether a nucleic acid increases the nuclear concentration of FKHR, a cell expressing a detectable cytoplasmic FKHR is employed. The detectable FKHR is typically modified to enable detection using an image based instrument platform. For example, a cell may be designed to express a recombinant FKHR protein containing a fluorescent protein tag.

A number of fluorescent proteins with various properties are commercially available. An important consideration is that the fluorescent properties of the protein should be compatible with the detection equipment such that it can be efficiently excited by the light source of the platform, and the emission wavelength can be detected. When the fluorescent protein is to be used as a marker of target protein translocation, it is important that the fluorescent protein does not itself direct the FKHR to a cellular compartment or cause oligomerization. The fluorescent protein should have strong fluorescence under the conditions tested, to minimize the number of molecules needed. In mammalian cells, EGFP may be desired.

Typically the cell employed is a stable clonal cell line expressing the FKHR interest fused to a fluorescent protein, such as GFP. Such cell lines have uniform expression of the GFP-fusion protein and the biological response is normally aligned within the cell population. Thus, image analysis may be performed in a robust and reproducible manner. Methods of developing clonal cell lines are well known in the art (see Sambrook et al., Molecular Cloning: A Laboratory Manual (2nd ed.). Cold Spring Harbor, N.Y.: Cold Spring Harbor Laboratory, 1989).

The phosphoinositide-3-kinase (PI3K) pathway may be activated when a ligand binds to a growth factor receptor (GFR) on the cell surface. In some cases, a cell may be employed that has a constitutively active AKT, such as a PTEN null cell. PI3K generates PIP3 in the plasma membrane, leading to translocation of Akt and other pH domain-containing proteins from the cytoplasm to the plasma membrane. Akt phosphorylates FKHR, resulting in export of the FKHR transcription factor from the nucleus to the cytoplasm. When the pathway is inhibited, for example, by addition of the PI3K inhibitor wortmannin, FKHR accumulates in the nucleus.

An image analysis algorithm can be used to quantify the translocation of FKHR between the nuclear and cytoplasmic compartments, as a measure of test nucleic acid activity. Image analysis algorithms typically require a nuclear marker in order to measure translocation. Hoechst stain is a very effective nuclear stain (added to the fixed cells as a 1 μM solution in PBS). DRAQ5 is another useful nuclear stain that has the advantage that it also stains RNA, though to a lesser extent than DNA. This allows it to be used as both a nuclear marker (intense staining) and a cytoplasmic marker (weaker staining), by using two different thresholds in the image analysis algorithm. DRAQ5 is used at a concentration of 0.3-1 μM, depending on cell density.

Image based instrument platforms are typically used for detection of the recombinant FKHR. A number of image-based instrument platforms for high content analysis are currently available, including GE Healthcare IN Cell 3000, Cellomics ArrayScan, Evotec Opera, CompuCyte ICyte, Molecular Devices Discovery 1, BD Biosciences Atto Pathfinder HT, and others. Manufacturers of the major imaging platforms provide standard algorithms with the instruments. Alternatively, it is possible for users with programming expertise to generate custom algorithms using programs such as MATLAB.

Thus, a query nucleic acid (test compound) may be added to the cell. If the query nucleic acid is capable of increasing the nuclear concentration of the FKHR protein, a decrease in the amount of cytoplasmic recombinant FKHR and increase in the nuclear recombinant FKHR is observed. The increase is relative to the amount of cytoplasmic recombinant FKHR and nuclear recombinant FKHR in the absence of query nucleic acid. Based on this method, the nuclear concentration to cytoplasmic concentration ratio may be ascertained for the query nucleic acid. In some embodiments, the addition of the query nucleic acid results in a nuclear concentration to cytoplasmic concentration ratio of at least 100 to 1,200 to 1,300 to 1,400 to 1,500 to 1,600 to 1,700 to 1,800 to 1,900 to 1, or 1000 to 1 when added at a concentration of no more than 50 nM, no more than 40 nM, less no more than 30 nM, no more than 20 nM, no more than 10 nM, no more than 5 nM, or no more than 2.5 nM. In some embodiments, the addition of the query nucleic acid results in a nuclear concentration to cytoplasmic concentration ratio of at least 100 to 1,200 to 1,300 to 1,400 to 1, or 500 to 1 when added at a concentration of about 10 nM, about 5 nM, or about 2.5 nM. In some embodiments, the addition of the query nucleic acid results in a nuclear concentration to cytoplasmic concentration ratio of about 150 to 1, 200 to 1, or 250 to 1 when added at a concentration of about 10 nM, about 5 nM, or about 2.5 nM.

To test whether a nucleic acid decrease cell viability or apoptosis, the change in cell density in the presence and absence of the query nucleic acid may be measured. Any appropriate method of measuring cell density may be used, such as microscopic methods of counting whole cells. Alternatively, a cell viability assay may be employed, such as the AlamarBlue™ assay, which incorporates a fluorometric/colometric indicator based on detection of metabolic activity. The system incorporates an oxidation-reduction (REDOX) indicator that can fluoresce and changes color. Thus, the cell viability in the presence or absence of query nucleic acid can be measured.

Generally, in cell based assays, cells are typically treated with the nucleic acid in a 96-well plate after reaching approximately 80% confluency. Nucleic acids may be introduced into cells using the cationic lipid transfection reagent LIPOFECTIN (Invitrogen Life Technologies, Carlsbad, Calif.). Nucleic acids are mixed with LIPOFECTIN in OPTI-MEM (Invitrogen Life Technologies, Carlsbad, Calif.) to achieve the desired final concentration of nucleic acid and LIPOFECTIN. Before adding the nucleic acid to the cells, nucleic acid, LIPOFECTIN and OPTI-MEM are mixed thoroughly and incubated for an appropriate amount of time. The plates are incubated for an appropriate amount of time after which the supernatant medium is removed and the Cells are analyzed or harvested after nucleic acid treatment, at which time the appropriate phenotypic assays performed. In general, data from treated cells are obtained in multiple trials, and results presented as an average of the trials.

In some embodiments, cells are transiently transfected with nucleic acid. The concentration of nucleic acid used varies from cell line to cell line. To determine the optimal nucleic acid concentration for a particular cell line, the cells are treated with a positive control nucleic acid at a range of concentrations.

Cell-based assays may involve whole cells or cell fractions. Exemplary cell types that can be used according to the methods and assays disclosed herein include, e.g., any mammalian cells including leukocytes such as neutrophils, monocytes, macrophages, eosinophils, basophils, mast cells, and lymphocytes, such as T cells and B cells, leukemias, Burkitt's lymphomas, tumor cells (including mouse mammary tumor virus cells), endothelial cells, fibroblasts, cardiac cells, muscle cells, breast tumor cells, ovarian cancer carcinomas, cervical carcinomas, glioblastomas, liver cells, kidney cells, and neuronal cells, as well as fungal cells, including yeast. Cells can be primary cells or tumor cells or other types of immortal cell lines.

A variety of useful assays for detecting hybridization of a nucleic acid to an RNA molecule in vitro are known in the art. Hybridization assays include, for example, Northern blots and RNase protection assays, and Southern blots. The nucleic acid or RNA molecule can be labeled with any suitable detectable moiety, such as a radioisotope, fluorochrome, chemiluminescent marker, biotin, or other detectable moiety known in the art that is detectable by analytical methods. High throughput methods employing biochip may be used to screen large populations of nucleic acids. The biochip may include a solid substrate with an attached nucleic acid or RNA molecule. The attached compounds may be at spatially defined addresses on the substrate. More than one nucleic acid or RNA molecule sequence may be used. The nucleic acids or RNA molecules may be attached to the biochip in a wide variety of ways, as will be appreciated by those in the art.

### VII. PHARMACEUTICAL COMPOSITIONS

The nucleic acid can be utilized in pharmaceutical compositions by adding an effective amount of the compound or composition to a suitable pharmaceutically acceptable diluent or carrier. The nucleic acids may also be useful prophylactically.

The nucleic acids and compositions may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative U.S. patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The pharmaceutical compositions may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The subject may be an animal or a human. An animal subject may be a mammal, such as a mouse, a rat, a dog, a guinea pig, a monkey, a non-human primate, a cat or a pig. Non-human primates include monkeys and chimpanzees. A suitable animal subject may be an experimental animal, such as a mouse, rat, mouse, a rat, a dog, a monkey, a non-human primate, a cat or a pig.

In some embodiments, an nucleic acid can be administered to a subject via an oral route of administration. Oral nucleic acid compositions may include one or more “mucosal penetration enhancers,” also known as “absorption enhancers” or simply as “penetration enhancers.” Accordingly, some embodiments include at least one nucleic acid in combination with at least one penetration enhancer. In general, a penetration enhancer is a substance that facilitates the transport of a drug across mucous membrane(s) associated with the desired mode of administration, e.g. intestinal epithelial membranes. Accordingly it is desirable to select one or more penetration enhancers that facilitate the uptake of one or more nucleic acids, without interfering with the activity of the compounds, and in such a manner the compounds can be introduced into the body of an animal without unacceptable side-effects such as toxicity, irritation or allergic response. Certain penetration enhancers have been used to improve the bioavailability of certain drugs. See Muranishi, Crit. Rev. Ther. Drug Carrier Systems, 1990, 7, 1 and Lee et al., Crit. Rev. Ther. Drug Carrier Systems, 1991, 8, 91.

Oral compositions for administration of non-parenteral nucleic acids and compositions may be formulated in various dosage forms such as, but not limited to, tablets, capsules, liquid syrups, soft gels, suppositories, and enemas. The term “alimentary delivery” encompasses e.g. oral, rectal, endoscopic and sublingual/buccal administration. A common requirement for these modes of administration is absorption over some portion or all of the alimentary tract and a need for efficient mucosal penetration of the nucleic acid(s) so administered.

Other excipients that may be added to oral nucleic acid compositions include surfactants (or “surface-active agents”), which are chemical entities which, when dissolved in an aqueous solution, reduce the surface tension of the solution or the interfacial tension between the aqueous solution and another liquid, with the result that absorption of nucleic acids through the alimentary mucosa and other epithelial membranes is enhanced. In addition to bile salts and fatty acids, surfactants include, for example, sodium lauryl sulfate, polyoxyethylene-9-lauryl ether and polyoxyethylene-20-cetyl ether (Lee et al., Critical Reviews in Therapeutic Drug Carrier Systems, 1991, page 92); and perfluorochemical emulsions, such as FC-43 (Takahashi et al., J. Pharm. Phamacol., 1988, 40, 252).

In some embodiments, nucleic acid compositions for oral delivery comprise at least two discrete phases, which phases may comprise particles, capsules, gel-capsules, microspheres, etc. Each phase may contain one or more nucleic acids, penetration enhancers, surfactants, bioadhesives, effervescent agents, or other adjuvant, excipient or diluent. In some embodiments, one phase comprises at least one nucleic acid and at least one penetration enhancer. In some embodiments, a first phase comprises at least one nucleic acid and at least one penetration enhancer, while a second phase comprises at least one penetration enhancer. In some embodiments, a first phase comprises at least one nucleic acid and at least one penetration enhancer, while a second phase comprises at least one penetration enhancer and substantially no nucleic acid. In some embodiments, at least one phase is compounded with at least one degradation retardant, such as a coating or a matrix, which delays release of the contents of that phase. In some embodiments, a first phase comprises at least one nucleic acid, at least one penetration enhancer, while a second phase comprises at least one penetration enhancer and a release-retardant. In particular embodiments, an oral nucleic acid comprises a first phase comprising particles containing an nucleic acid and a penetration enhancer, and a second phase comprising particles coated with a release-retarding agent and containing penetration enhancer.

A variety of bile salts also function as penetration enhancers to facilitate the uptake and bioavailability of drugs. The physiological roles of bile include the facilitation of dispersion and absorption of lipids and fat-soluble vitamins (Brunton, Chapter 38 In: Goodman & Gilman's The Pharmacological Basis of Therapeutics, 9th Ed., Hardman et al., eds., McGraw-Hill, New York, N.Y., 1996, pages 934-935). Various natural bile salts, and their synthetic derivatives, act as penetration enhancers. Thus, the term “bile salt” includes any of the naturally occurring components of bile as well as any of their synthetic derivatives. The bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (CDCA, sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1; Yamamoto et al., J. Pharm. Exp. Ther., 1992, 263, 25; Yamashita et al., J. Pharm. Sci., 1990, 79, 579).

Other excipients include chelating agents, i.e. compounds that remove metallic ions from solution by forming complexes therewith, with the result that absorption of nucleic acids through the alimentary and other mucosa is enhanced. With regard to their use as penetration enhancers, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315). Chelating agents include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 1990, 7, 1; Buur et al., J. Control Rel., 1990, 14, 43).

Some oral nucleic acid compositions also incorporate carrier compounds in the formulation. As used herein, “carrier compound” or “carrier” can refer to a nucleic acid, or analog thereof, which may be inert (i.e., does not possess biological activity per se) or may be necessary for transport, recognition or pathway activation or mediation, or is recognized as a nucleic acid by in vivo processes that reduce the bioavailability of an nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate nucleic acid in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115; Takakura et al., Antisense & Nucl. Acid Drug Dev., 1996, 6, 177).

A “pharmaceutical carrier” or “excipient” may be a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with an nucleic acid and the other components of a given pharmaceutical composition. Typical pharmaceutical carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, EXPLOTAB); and wetting agents (e.g., sodium lauryl sulphate, etc.).

For topical or other administration, nucleic acids and compositions may be encapsulated within liposomes or may form complexes thereto, in particular to cationic liposomes. Alternatively, they may be complexed to lipids, in particular to cationic lipids. Topical formulations are described in detail in U.S. patent application Ser. No. 09/315,298 filed on May 20, 1999, which is incorporated herein by reference in its entirety.

In another embodiment, nucleic acid compositions may contain one or more of the anti-miRNA nucleic acids and compositions targeted to a first miRNA target and one or more additional nucleic acids targeted to a second miRNA target. Alternatively, compositions may contain two or more nucleic acids and compositions targeted to different regions, segments or sites of the same miRNA target. Two or more combined compounds may be used together or sequentially.

A pharmaceutical composition can be micronized or powdered so that it is more easily dispersed and solubilized by the body. Processes for grinding or pulverizing drugs are well known in the art, for example, by using a hammer mill or similar milling device.

Dosage forms (compositions) suitable for internal administration contain from about 1.0 milligram to about 5000 milligrams of active ingredient per unit. In these pharmaceutical compositions, the active ingredient may be present in an amount of about 0.5 to about 95% by weight based on the total weight of the composition. Another convention for denoting the dosage form is in mg per meter squared (mg/m²) of body surface area (BSA). Typically, an adult will have approximately 1.75 m² of BSA. Based on the body weight of the patient, the dosage may be administered in one or more doses several times per day or per week. Multiple dosage units may be required to achieve a therapeutically effective amount. For example, if the dosage form is 1000 mg, and the patient weighs 40 kg, one tablet or capsule will provide a dose of 25 mg per kg for that patient. It will provide a dose of only 12.5 mg/kg for a 80 kg patient.

By way of general guidance, for humans a dosage of as little as about 0.25 milligrams (mg) per kilogram (kg) of body weight and up to about 600 mg per kg of body weight is suitable as a therapeutically effective dose. In certain embodiments, from about 1 mg/kg to about 600 mg/kg of body weight is used. Other embodiments include doses range from 50 mg/kg to about 600 mg/kg of body weight, from 100 mg/kg to about 600 mg/kg of body weight, from 200 mg/kg to about 600 mg/kg of body weight, or from 300 mg/kg to about 500 mg/kg of body weight. In some embodiments, a dosage of about 400 mg per kg of body weight is employed.

Intravenously, the certain rates of administration can range from about 1 to about 1000 mg/kg/minute during a constant rate infusion. A pharmaceutical composition can be administered in a single daily dose, or the total daily dosage may be administered in divided doses of two, three, or four times daily. A nucleic acid is generally given in one or more doses on a daily basis or from one to three times a week.

A pharmaceutical composition may be administered by any conventional means available for use in conjunction with pharmaceuticals, either as individual therapeutic agents or in combination with other therapeutic agents.

In another aspect, a pharmaceutical kit is provided. The pharmaceutical kit is useful, for example, for the treatment of cancer, which comprise one or more containers containing a pharmaceutical composition comprising a therapeutically effective amount of a nucleic acid. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit. It should be understood that although the specified materials and conditions are important in practicing the methods described herein, unspecified materials and conditions are not excluded so long as they do not prevent the benefits of the methods from being realized.

The terms and expressions which have been employed herein are used as terms of description and not of limitation, and there is no intention in the use of such terms and expressions of excluding equivalents of the features shown and described, or portions thereof, it being recognized that various modifications are possible within the scope of the subject matter claimed. Moreover, any one or more features of any embodiment of the present subject matter may be combined with any one or more other features of any other embodiment of the subject matter where appropriate, without departing from the scope of the subject matter described herein. For example, the applicable features of the methods of increasing apoptosis in a cell are equally applicable to the methods of treating cancer.

### VIII. EXAMPLES

The following examples are meant to merely illustrate certain embodiments of the technology disclosed herein, and are not meant to limit the scope of the invention.

### Example 1

### Identification of Nucleic Acids

A library of 288 sequence-specific anti-miRNA nucleic acids were synthesized using locked nucleic acids (LNA) phosphoramidites. The general procedure employed for the synthesis of LNA oligonucleotides containing phosphodiester internucleotide linkages is set forth below. LNA synthesis was performed on one of the following solid-phase synthesizers using LNA phosphoramidites purchased from Sigma/PrOligo: Applied Biosystems model ABI 3900 or ABI 394 or MerMase-12. Oligonucleotide chains were built on 3′-dT-column support using iterative cycles of deprotection/activation/coupling and oxidation to form phosphodiester internucleotide linkages. After the final coupling the 5′-dimethoxytrityl protection group was left on to facilitate subsequent purification by solid phase extraction on C-18 column support. The anti-miRNA nucleic acid library was designed to target a collection of 369 human miRNA sequences by perfect complimentary base pairing (see Table 1). Nucleic acid sequences bearing LNA chemistry provides high-affinity binding to their complimentary miRNA sequences, and provide nuclease stability towards this class of miRNA antagonist. The LNA-based anti-miRNA nucleic acid library was targeted against approximately 80% of the known human miRNAs.

These LNA-based anti-miRNA nucleic acids were synthesized using standard solid phase nucleic acid synthesis and arrayed in 96-well plates for cell-based phenotypic screening. PTEN null MDA-MB-468 cells were employed, in which FKHR is located in the cytoplasm due to constitutive AKT activity. A human breast adenocarcinoma cell line, MDA-468, was obtained from ATCC (ATCC number: HTB-132) and maintained in RPMI with 10% fetal bovine serum at 37° C. with 5% CO₂. The PTEN null MDA-MB-468 cells were further engineered to carry GFP-tagged FKHR in the miRNA interference screen. The MDA-MB-468-FKHRL1-dGPF cell line was generated upon the transfection with a pcDNA1.1 mammalian expression plasmid containing human FKHRL1 cDNA with dGFP (from Diversa Inc.) cDNA fused to its C-terminus, and a zeocin-resistant gene. Stable cell lines expressing FKHRL1-dGFP fusion protein were selected by treating transfected cells with 200 μg/ml zeocin. The expression of FKHRL1-dGFP was verified by nuclear translocation of the fluorescent protein induced by PI3-kinase inhibitor wortmannin. The presence of GFP-tag allowed facile detection of sub-cellular localization of FKHR upon the introduction of miRNA inhibitors in high-content image analysis

MDA-MB-468 cells were seeded at a concentration of 2500 cells/well in 96-well plates. After approximately 24 hours, cells in inner 60-wells were transfected with a unique LNA sequence from the synthetic library using 0.19% Lipofectamin2000 (from Invitrogen) according to the manufacturer's procedure. In the primary screen carried out with the complete synthetic LNA library, duplicate cell plates were included in which any given LNA transfection was duplicated on two plates at 65 nM LNA concentration. This initial screen identified potential LNA sequences (initial hits) that induced nuclear translocation of FKHDR. In subsequent screens, these initial hit LNA sequences were transfected at 20, 10 & 5, and 2.5 nM to rank their potency in inducing nuclear translocation of FKHR and decreasing cell viability.

After 72 hrs, cells were fixed in 10% PBS containing 3.7% formaldehyde and 5 μg/mL Hoechst nuclear stain for 30 min at ambient temperature. Cells were washed twice with 150 μL of PBS and stored in PBS at 4° C. until image analysis. Image analysis was performed on a Cellomics Array Scan that quantifies GFP fluorescence intensity in the cytoplasm and in the nucleus. Array Scan algorithm discriminates the nuclear area from the cytoplasm using the Hoechst fluorescence of a distinct wavelength that stains the nucleus. A typical Array Scan reading of nuclear and cytoplasmic fluorescence represents an average of fluorescence derived from a survey of four non-overlapping areas in a single well. The cell number reflects the average reading of the number of nuclear events in four different areas in a single well. The parameter Nucl/Cyt indicates the ratio of GFP fluorescence intensity in the nucleus to that in the cytoplasm. Image analysis was performed in Cellomics Array scan to evaluate the difference of GFP signal between the nucleus and the cytoplasm as well as the cell number in each well. For each screen, duplicate plates were run and each inhibitor was introduced into duplicate wells on each plate. Image analysis data, averaged over 4 wells (intra- & inter-plates) per anti-miRNA nucleic acid were used in Spotfire software for hit identification.

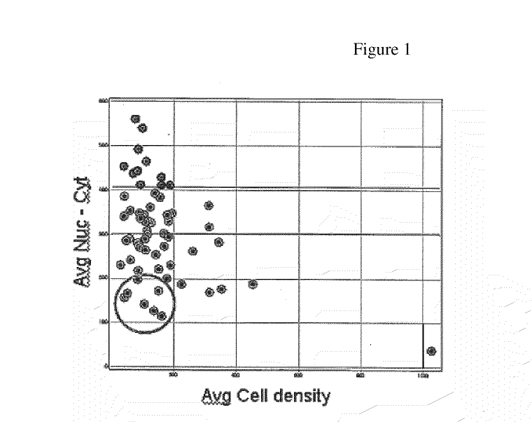

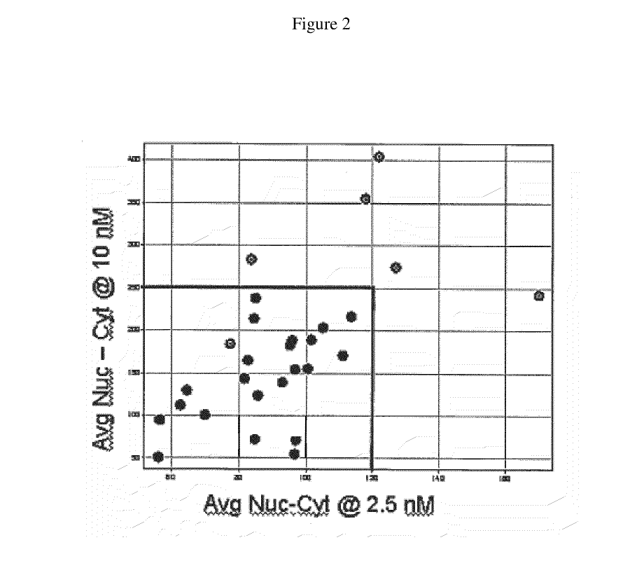

FIG. 1 sets forth an example of data analysis from an experiment in which 65 nM of anti-miRNA nucleic acid was employed. A plot of the ratio of FKHR concentration in the nucleus to FKHR concentration in the cytoplasm versus cell density (an indicator of cell death) is shown. FIG. 2 shows a representative example of data analysis from an experiment in which 10 nM and 2.5 nM of anti-miRNA nucleic acid was employed. The ratio of FKHR concentration in the nucleus to FKHR concentration in the cytoplasm at nucleic acid concentrations of 2.5 nM and 10 nM is set forth.

From the initial 288 query nucleic acids, fifteen anti-miRNA nucleic acids were identified that increased nuclear concentration of FKHR protein and decreased cell viability. The anti-miRNA portion of the identified nucleic acids are set forth in Table 2. Due to the use of a dT column during synthesis, the nucleic acids identified in this Example 1 consist of the stated sequences in Table 2 and a deoxy-T at the 3′ end of the sequences. One of skill in the art will immediately recognize that SEQ ID Nos: 1 to 15 per se do not include the 3′ dT, and where one of SEQ ID Nos: 1 to 15 are claimed or referred to within Sections I to VII above, the 3′ dT is not intended to be included in the nucleic acid sequence. The sequences of the target miRNAs are provided in Table 3.

TABLE 1

| miRNA | miRNA | miRNA | miRNA | miRNA | miRNA |
|---|---|---|---|---|---|
| let-7a-1 | miR-31 | miR-147 | miR-216 | miR-380-3p | miR-514-2 |
| let-7a-2 | miR-32 | miR-148a | miR-217 | miR-380-5p | miR-514-3 |
| let-7a-3 | miR-33 | miR-148b | miR-218-1 | miR-381 | miR-515-1-3p |
| let-7b | miR-34a | miR-149 | miR-218-2 | miR-382 | miR-515-2-3p |
| let-7c | miR-34b | miR-150 | miR-219-1 | miR-383 | miR-515-1-5p |
| let-7d | miR-34c | miR-151 | miR-219-2 | miR-384 | miR-515-2-5p |
| let-7e | miR-92-1 | miR-152 | miR-220 | miR-409-3p | miR-516-1-3p |
| let-7f-1 | miR-92-2 | miR-153-1 | miR-221 | miR-409-5p | miR-516-2-3p |
| let-7f-2 | miR-93 | miR-153-2 | miR-222 | miR-410 | miR-516-3-3p |
| let-7g | miR-95 | miR-154 | miR-223 | miR-412 | miR-516-4-3p |
| let-7i | miR-96 | miR-154* | miR-224 | miR-422a | miR-516-1-5p |
| miR-1-1 | miR-98 | miR-155 | miR-296 | miR-422b | miR-516-2-5p |
| miR-1-2 | miR-99a | miR-181a | miR-299-3p | miR-423 | miR-517-a* |
| miR-7-1 | miR-99b | miR-181b-1 | miR-299-5p | miR-424 | miR-517-b* |
| miR-7-2 | miR-100 | miR-181b-2 | miR-301 | miR-425 | miR-517-c* |
| miR-7-3 | miR-101-1 | miR-181c | miR-302a | miR-429 | miR-517a |
| miR-9-1 | miR-101-2 | miR-181d | miR-302b | miR-431 | miR-517b |
| miR-9-2 | miR-103-1 | miR-182 | miR-302c | miR-432 | miR-517c |
| miR-9-3 | miR-103-2 | miR-182* | miR-302d | miR-432* | miR-518a-1 |
| miR-9*-3 | miR-105-1 | miR-183 | miR-302a* | miR-433 | miR-518a-2 |
| miR-9*-1 | miR-105-2 | miR-184 | miR-302b* | miR-448 | miR-518b |
| miR-9*-2 | miR-106a | miR-185 | miR-302c* | miR-449 | miR-518c |
| miR-10a | miR-106b | miR-186 | miR-320 | miR-450-1 | miR-518d |
| miR-10b | miR-107 | miR-187 | miR-323 | miR-450-2 | miR-518e |
| miR-15a | miR-122a | miR-188 | miR-324-3p | miR-451 | miR-518f |
| miR-15b | miR-124a-1 | miR-189 | miR-324-5p | miR-452 | miR-518a-2* |
| miR-16-1 | miR-124a-2 | miR-190 | miR-325 | miR-452* | miR-518c* |
| miR-16-2 | miR-124a-3 | miR-191 | miR-326 | miR-453 | miR-518f* |
| miR-17-3p | miR-125a | miR-191* | miR-328 | miR-485-3p | miR-519a-1 |
| miR-17-5p | miR-125-b-1 | miR-192 | miR-329-1 | miR-485-5p | miR-519a-2 |
| miR-18a | miR-125-b-2 | miR-193a | miR-329-2 | miR-488 | miR-519b |
| miR-18b | miR-126 | miR-193b | miR-330 | miR-489 | miR-519c |
| miR-19a | miR-126* | miR-194-1 | miR-331 | miR-490 | miR-519d |
| miR-19b-1 | miR-127 | miR-194-2 | miR-335 | miR-491 | miR-519e |
| miR-19b-2 | miR-128a | miR-195 | miR-337 | miR-492 | miR-519e* |
| miR-20a | miR-128b | miR-196a-1 | miR-338 | miR-493 | miR-520a |
| miR-20b | miR-129-1 | miR-196a-2 | miR-339 | miR-494 | miR-520b |
| miR-21 | miR-129-2 | miR-196b | miR-340 | miR-495 | miR-520c |
| miR-22 | miR-130a | miR-197 | miR-342 | miR-496 | miR-520d |
| miR-23a | miR-130b | miR-198 | miR-345 | miR-497 | miR-520e |
| miR-23b | miR-132 | miR-199a-1 | miR-346 | miR-498 | miR-520f |
| miR-24-1 | miR-133a-1 | miR-199a*-1 | miR-361 | miR-499 | miR-520g |
| miR-24-2 | miR-133a-2 | miR-199a-2 | miR-362 | miR-500 | miR-520h |
| miR-25 | miR-133b | miR-199a*-2 | miR-363 | miR-501 | miR-520a* |
| miR-26a-1 | miR-134 | miR-199b | miR-365-1 | miR-502 | miR-520d* |
| miR-26a-2 | miR-135a-1 | miR-200a | miR-365-2 | miR-503 | miR-521-1 |
| miR-26b | miR-135a-2 | miR-200b | miR-367 | miR-504 | miR-521-2 |
| miR-27a | miR-135b | miR-200c | miR-368 | miR-505 | miR-522 |
| miR-27b | miR-136 | miR-200a* | miR-369-3p | miR-506 | miR-523 |
| miR-28 | miR-137 | miR-202 | miR-369-5p | miR-507 | miR-524 |
| miR-29a | miR-138-1 | miR-202* | miR-370 | miR-508 | miR-524* |
| miR-29b-1 | miR-138-2 | miR-203 | miR-371 | miR-509 | miR-525 |
| miR-29b-2 | miR-139 | miR-204 | miR-372 | miR-510 | miR-525* |
| miR-29c | miR-140 | miR-205 | miR-373 | miR-511-1 | miR-526c |
| miR-30a-3p | miR-141 | miR-206 | miR-373* | miR-511-2 | miR-526a |
| miR-30a-5p | miR-142-3p | miR-208 | miR-374 | miR-512-1-3p | miR-526b |
| miR-30b | miR-142-5p | miR-210 | miR-375 | miR-512-2-3p | miR-526b* |
| miR-30c-1 | miR-143 | miR-211 | miR-376a | miR-512-1-5p | miR-527 |
| miR-30c-2 | miR-144 | miR-212 | miR-376b | miR-512-2-5p | miR-30e-5p |
| miR-30d | miR-145 | miR-213 | miR-377 | miR-513-1 | miR-146b |
| miR-30e-3p | miR-146a | miR-214 | miR-378 | miR-513-2 | miR-215 |
| miR-379 | miR-514-1 | | | | |

TABLE 2

| | | |
|---|---|---|
| Nucleic acid | Sequence | miRNA Target |

| | | |
|---|---|---|
| SEQ ID NO:1 | ACUGGUACAAGGGUUGGGAGA | miRNA-150 |
| SEQ ID NO:2 | GGCAUAGGAUGACAAAGGGAA | miRNA-204 |
| SEQ ID NO:3 | CCAGCUAACAAUACACUGCCA | miRNA-449 |
| SEQ ID NO:4 | AUUAGGAACACAUCGCAAAAA | miRNA-450-1 |
| SEQ ID NO:5 | UACUCCAAAAGGCUACAAUCA | miRNA-508 |
| SEQ ID NO:6 | GCCUUCUGACUCCAAGUCCAG | miRNA422b |
| SEQ ID NO:7 | GGCGAAGGAUGACAAAGGGAA | miRNA-211 |
| SEQ ID NO:8 | CGGAAGGGCAGAGAGGGCCAG | miRNA-328 |
| SEQ ID NO:9 | UACCCCUGGAGAUUCUGAUAA | miRNA-361 |
| SEQ ID NO:10 | CAGACCGAGACAAGUGCAAUG | miRNA-25 |
| SEQ ID NO:11 | AUCAGCUAAUGACACUGCCUA | miRNA-34b |
| SEQ ID NO:12 | GCUCAAUAAAUACCGUUGAA | miRNA-95 |
| SEQ ID NO:13 | GCAAGGUCGGUUCUACGGGUG | miRNA-99b |
| SEQ ID NO:14 | ACCUGCACUGUAAGCACUUUG | miRNA-17-5p |
| SEQ ID NO:15 | GAAGGCAACACGGAUAACCUA | miRNA-154 |

The sequences in Table 2 are in the 5′ to 3′ direction.

TABLE 3

| | |
|---|---|
| miRNA Target | Sequence |

| | | |
|---|---|---|
| miRNA-150 | (SEQ ID NO:16) | UCUCCCAACCCUUGUACCAGUG |
| miRNA-204 | (SEQ ID NO:17) | UUCCCUUUGUCAUCCUAUGCCU |
| miRNA-449 | (SEQ ID NO:18) | UGGCAGUGUAUUGUUAGCUGGU |
| miRNA-450-1 | (SEQ ID NO:19) | UUUUUGCGAUGUGUUCCUAAUA |
| miRNA-508 | (SEQ ID NO:20) | UGAUUGUAGCCUUUUGGAGUAGA |
| miRNA422b | (SEQ ID NO:21) | CUGGACUUGGAGUCAGAAGGCC |
| miRNA-211 | (SEQ ID NO:22) | UUCCCUUUGUCAUCCUUCGCCU |
| miRNA-328 | (SEQ ID NO:23) | CUGGCCCUCUCUGCCCUUCCGU |
| miRNA-361 | (SEQ ID NO:24) | UUAUCAGAAUCUCCAGGGGUAC |
| miRNA-25 | (SEQ ID NO:25) | CAUUGCACUUGUCUCGGUCUGA |
| miRNA-34b | (SEQ ID NO:26) | UAGGCAGUGUCAUUAGCUGAUUG |
| miRNA-95 | (SEQ ID NO:27) | UUCAACGGGUAUUUAUUGAGCA |
| miRNA-99b | (SEQ ID NO:28) | CACCCGUAGAACCGACCUUGCG |
| miRNA-17-5p | (SEQ ID NO:29) | CAAAGUGCUUACAGUGCAGGUAGU |
| miRNA-154 | (SEQ ID NO:30) | UAGGUUAUCCGUGUUGCCUUCG |

The sequences in Table 3 are in the 5′ to 3′ direction. The underlined portions highlight the seed region of the miRNA.

### Example 2

### Specificity in Nuclear Translocation Process

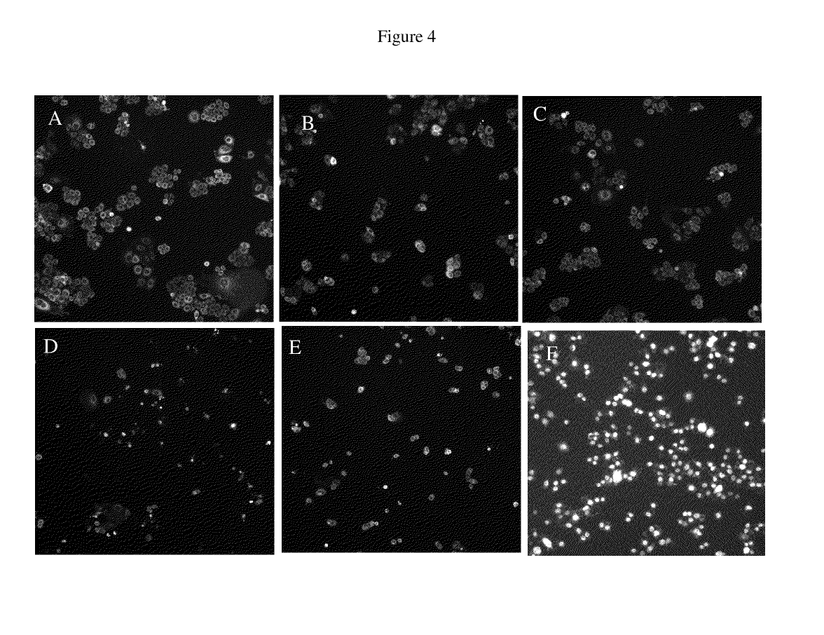

Cellular images of MDA-468 cells bearing GFP-FKHR fusion protein 48 hrs after transfection of LNA inhibitors to selected miRNAs are shown in FIG. 4. The images were produced using the Cellomics Array Scan. Strausporin (25 mM, 1 hr treatment) provided a positive control that induces the nuclear translocation of FKHR by inhibiting many protein kinases, including AKT-1, -2 & -3. An anti-miRNA LNA nucleic acid perfectly complementary to miR-16 containing a 3′ deoxythymidine provided a negative control. The localization of GFP fluorescence is indicated by the light color in these images. The GFP fluorescence is predominantly localized in either cytoplasm (Untreated, Mock (lipofectin only), and miRNA-16) or nucleus (miRNA-204, miRNA-508 & Strausporin). The GFP-FKHDR fusion protein in MDA-468 cells was localized in the nucleus upon the transfection with LNA inhibitors to miR-204, miR-508. In contrast, inhibition of a control miRNA, miR-16, using the corresponding LNA sequence did not induce the nuclear localization of GFP-FKHDR (compare upper & lower left panels), demonstrating the specificity of the selected miRNAs in the nuclear translocation process.

### Example 3

### Cell Viability Effect in Different Cell Types

Additional cell lines were also tested using certain anti-miRNA nucleic acids in Table 2. MiaPaCa2, T24, or HeLa were plated in 96-well plates at a density of 7500/well 24 hrs before transfection of an anti-miRNA nucleic acid sequence (Example 1, Table 2) at varying concentrations using 0.19% of Lipofectamin2000 according to manufacturer's instructions. Seventy two hours after transfection, the cell viability was assessed by adding AlamarBlue™ (from Biosource; 10 μL/well). After incubation at 37° C. for 1-2 hr (the optimal time is empirically determined for each cell line), the fluorescence was measured at 590 nm using a Wallac Victor2 multi-well plate reader. AlamarBlue™ is reduced to a fluorogenic compound by viable cells and the amount of fluorescence in each well is a measure of the live cell content. In each case, the % viability was calculated using the AlamarBlue™ readings of wells transfected with anti-miRNA nucleic acid sequence normalized to the reading derived from wells subjected to mock transfection with Lipofectimine2000.

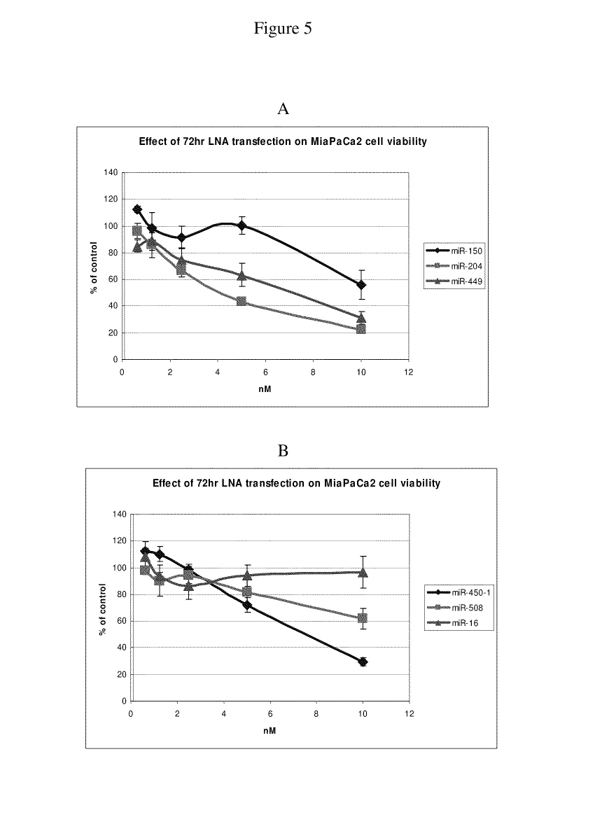

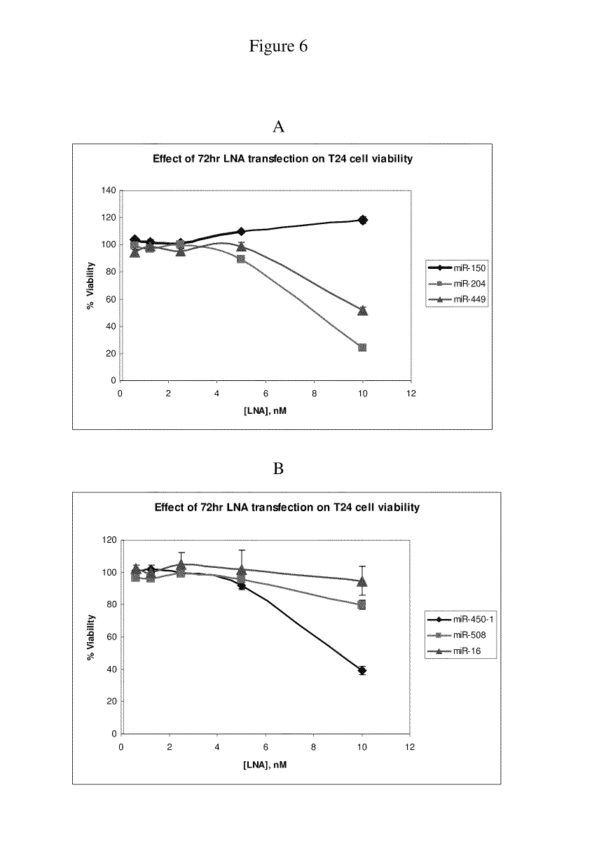

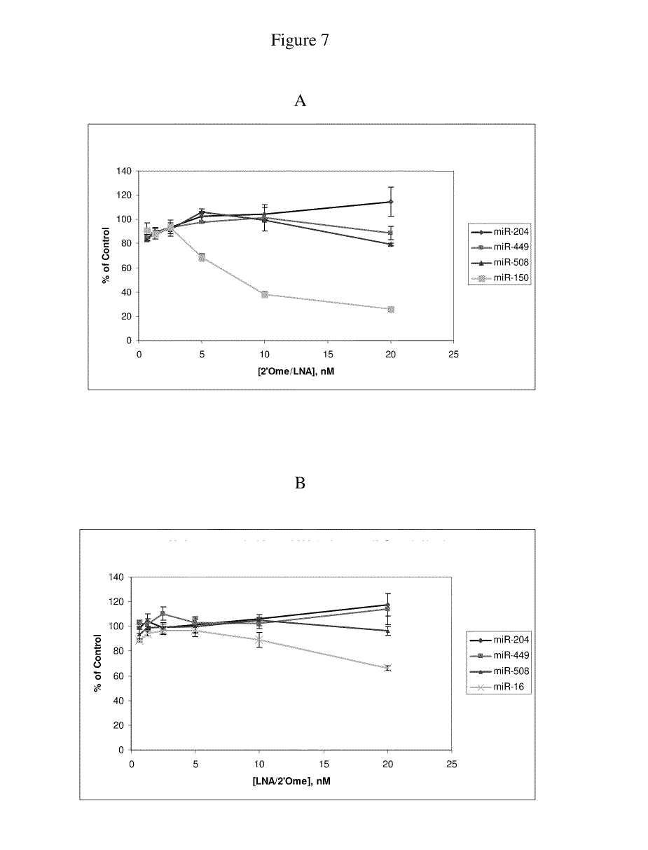

Results are presented in FIGS. 5, 6, and 7. In MiaPaca2 cell line, the inhibition of miRNA-150, miRNA-204, miRNA-449, miRNA-450-1 and miRNA-508 using the appropriate anti-mRNA sequences (as set forth in Table 2 and Example 1) resulted in a reduced number of cells 72 hrs after transfection. See FIG. 5, panels A and B. In T24 cells, nucleic acids targeting miRNA-204, miRNA-449 and miRNA-450-1 decreased the number of cells 72 hrs after transfection. See FIG. 6, panels A and B. In HeLa cells, nucleic acids targeting miRNA-150 decreased the number of cells 72 hrs after transfection. See FIG. 7, panel A. miRNA-16 is a negative control nucleic acid that is perfectly complementary LNA sequence (containing a 3′ deoxythymidine) to miRNA-16. See FIG. 5, panel B, and FIG. 6, panel B, and FIG. 7, panel B.

### Example 4

### Nucleic Acid Chemical Modification Study

Four different chemically modified nucleic acids were synthesized based on the sequence of selected anti-miRNA nucleic acids. The first modification (LNA) contains the same chemical modifications as described above in Example 1, which include all LNA nucleotides (except for the deoxythymidine at the 3′ end), and phosphodiester internucleotide linkages. The second modification (LNA/DNA) includes an LNA at every third nucleotide from the 5′ end, where the remaining nucleotides are DNA, and all internucleotide linkages are phosphorothioate linkages. The third modification (OMe/DNA) includes a 2′-O-methyl nucleotide every third nucleotide from the 5′ end, where the remaining nucleotides are DNA, and all internucleotide linkages are phosphorothioate linkages. The fourth modification (LNA/OMe) includes an LNA every third nucleotide from the 5′ end, where the remaining nucleotides are 2′-O-methyl, and all internucleotide linkages are phosphorothioate linkages.

The LNA, LNA/DNA, OMe/DNA, and LNA/OMe sequences are set forth below, where the asterisk (*) represents a phosphorothioate linkage. Small cap in the LNA sequence and the LNA/DNA sequence represents a deoxyribonucleotide; small cap in the OME/DNA sequence represents a 2′-O-methyl nucleotide; and a small cap in LNA/OMe represents an LNA unit with the exception of the 3′ deoxythymidine:

LNA:

(SEQ ID NO:31)

5′-A-C-U-G-G-U-A-C-A-A-G-G-G-U-U-G-G-G-A-G-A-t-3′
LNA/DNA:

(SEQ ID NO:32)

5′-u*a*C*a*a*G*g*g*U*u*g*G*g*a*G*a*t-3′
OMe/DNA:

(SEQ ID NO:33)

5′-u*a*C*a*a*G*g*g*U*u*g*G*g*a*G*a*T-3′
LNA/OMe:

(SEQ ID NO:34)

5′-u*a*C*a*a*G*g*g*U*u*g*G*g*a*G*a*t-3′.

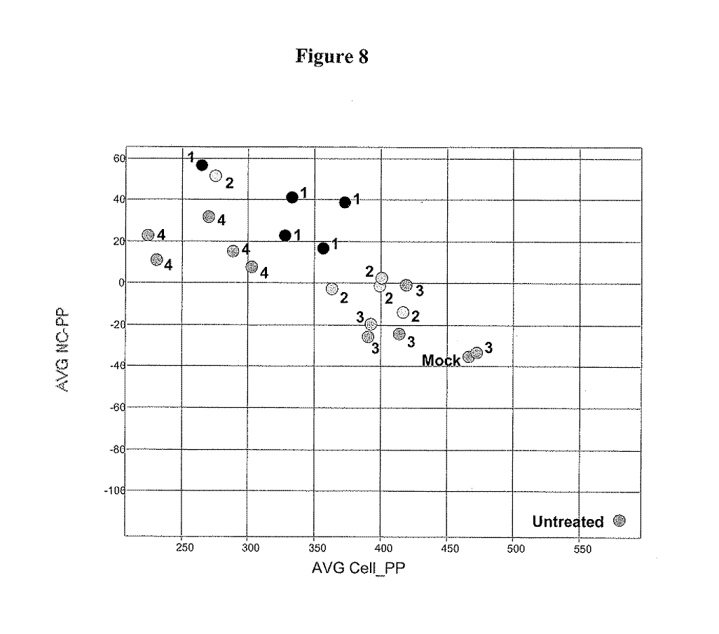

Results are shown in FIG. 8, which plots the ratio of nuclear FKHR to cytoplasmic FKHR (AVG NC-PP) versus cell density (AVG Cell_PP), as set forth in the methods provided above. Integer 1 denotes LNA sequences complementary to miRNA-422b, miRNA-150, miRNA-204, miRNA 449, and miRNA-508; integer 2 denotes LNA/DNA sequences complementary to miRNA-422b, miRNA-150, miRNA-204, miRNA 449, and miRNA-508; integer 3 denotes OMe/DNA sequences complementary to miRNA-422b, miRNA-150, miRNA-204, miRNA 449, and miRNA-508; and integer 4 denotes LNA/OMe sequences complementary to miRNA-422b, miRNA-150, miRNA-204, miRNA 449, and miRNA-508. Based on the nuclear translocation efficacy, the LNA and LNA/OMe nucleic acids were generally superior to the LNA/DNA and OMe/DNA nucleic acids. Sequences in the second, third, and fourth modifications are relatively shorter (17-mers, neglecting the common 3′-dT) than the complete LNA with natural phosphodiester backbone. Thus, sequences shorter than 21 nucleotides may be effective in the methods disclosed herein.

### Example 5

### Uptake of Cholesterol Conjugated Nucleic Acids

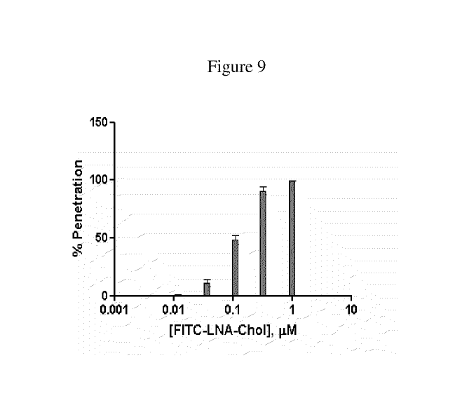

In this experiment, various conjugated forms of the nucleic acid inhibitors were prepared to identify parameters that promote cellular uptake. For instance, to facilitate the delivery of oligonucleotide-based miRNA inhibitors into cells, LNA/OMe chimeric sequences carrying a cholesterol moiety at the 3′ end and fluorescein at the 5′ end were synthesized. The cell lines were contacted with different constructs in culture. The constructs were taken up by both adherent and suspension cells in a concentration dependent manner. For example, in HeLa cells a mono-cholesterol-conjugated LNA/OMe sequence localized into intracellular foci by 48 hrs after incubation with cells and remained localized after 72 hr. FIG. 9 illustrates the uptake of nucleic acid sequences conjugated to cholesterol and fluorescein by different adherent cells. The amount of intracellular fluorescence is shown as a function of the concentration of nucleic acid conjugated to fluorescein and cholesterol.

In other experiments, the uptake of a bis-cholesterol conjugated LNA/OMe chimeric oligonucleotide carrying cholesterol at each end displayed a more efficient intracellular uptake than that of the mono-cholesterol counterpart. Even at 6 hr after incubation, the bis-cholesterol derivative was localized into concentrated areas and there was a significant amount left inside cells even at 72 hr. These experiments demonstrate that cholesterol conjugation is an example of one effective means to promote uptake of nucleic acid inhibitors such as those identified herein.

### Example 6

### Cell Cycle Effects of miRNA Inhibitors

As demonstrated above, cells exhibited compromised cell viability when incubated with inhibitors to the miRNAs identified to inhibit nuclear translocation of FKHR. To further understand the biological consequences of these inhibitors, a FACS (Fluorescence Activated Cell Sorting)-based cell cycle analysis was performed on cells incubated with inhibitors with or without cholesterol conjugation.

Cell cycle analysis information and caspase 3 measurements were obtained by growing cells in 96-well tissue culture plate at a density of about 10,000 cells/well and treating the cells with an anti-miRNA nucleic acid as provided herein. The concentration of the nucleic acid ranged from 0.01 to 10 μM. The cells were fixed ice-cold 90% methanol. Cells were washed with 200 μL/well in a wash buffer consisting of 1% BSA and 0.2% Triton-X100 in PBS prior to the incubation in 100 μL of 0.5% Triton-X100 in 2N HCl for 1 hr at room temperature in dark. These cells were washed several times in the wash buffer to reach neutral pH. Cells were then incubated in 80 μL of wash buffer and 20 μL of FITC-conjugated antibody against caspase-3 (BD Bioscience, Catalogue number 51-68654) at 3 μg/mL for 2 hr at room temperature in dark. After washing once in the wash buffer, cells were stored in 200 μL of PI (Propidium Iodide) staining buffer (BD Bioscience, Catalogue number 55-0825) until FACS analysis carried out using BD LSRII FACS machine.

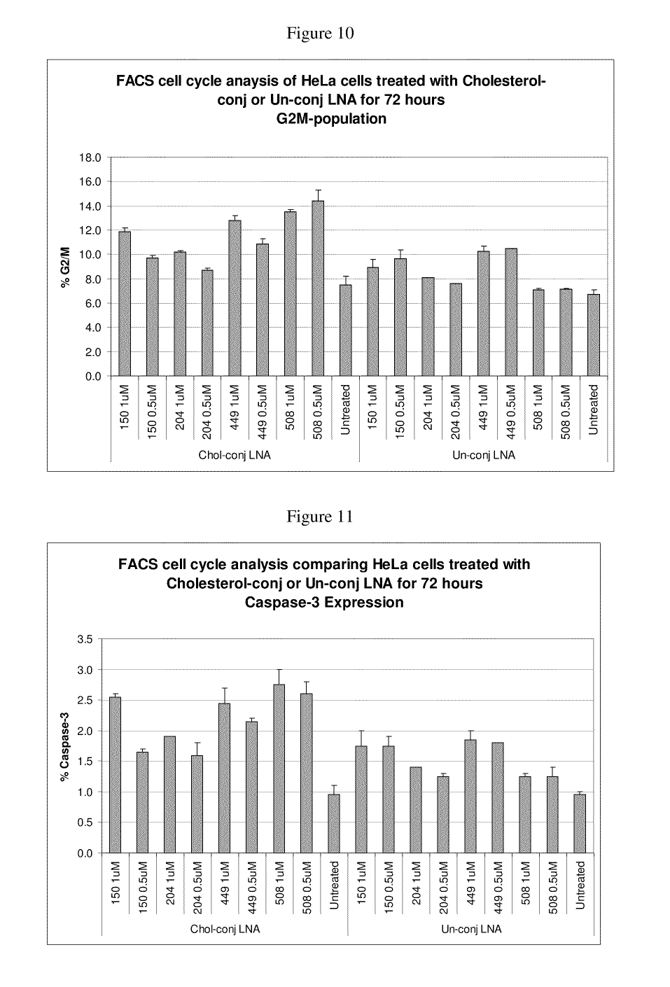

FIG. 10 illustrates a cell cycle analysis of cells treated with nucleic acid sequence inhibitors with (left) or without (right) cholesterol conjugation. The percentage of G2/M cell population in each case is shown. Compared to untreated cells, cells treated with an anti-miRNA nucleic acid provided herein led to an increase in G2/M cell population (see FIG. 10, Un-conj LNA), indicating that they are capable of inhibiting cell proliferation by modulating cell cycle progression. This effect was more pronounced with inhibitors carrying the cholesterol conjugation (see FIG. 10, Chol-conj LNA), which is in agreement with enhanced cell penetrating efficiency associated with cholesterol-conjugated oligonucleotides.

Referring to FIG. 11, anti-miRNA nucleic acids provided herein are capable of activating caspase-3, a mediator of intrinsic apoptotic process that responds to intracellular stimuli to trigger cell death. FIG. 11 illustrates an analysis of caspase 3 levels in cells treated with nucleic acid inhibitors with (left) or without (right) cholesterol conjugation. Compared to untreated cells, cells treated with an anti-miRNA nucleic acid provided herein led to an increase in caspase 3 levels (see FIG. 11, Un-conj LNA). This effect was more pronounced with inhibitors carrying the cholesterol conjugation (see FIG. 11, Chol-conj LNA). These results indicate that anti-miRNA nucleic acids are capable of modulating apoptosis and cell cycle progression. Modulating these processes may contribute to the overall decrease in viability of cells incubated with inhibitors to selected miRNAs.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes to the same extent as if each individual publication, patent or patent application were specifically and individually indicated to be so incorporated by reference.

What is claimed is:

1. A method of decreasing cell viability, said method comprising introducing into said cell a nucleic acid hybridizable to an RNA molecule, wherein:
(a) said RNA molecule is selected from the group consisting of miRNA-150, miRNA-204, miRNA-449, miRNA-450-1, miRNA-508, and precursors thereof; and
(b) said nucleic acid (i) hybridizes under stringent conditions to said RNA molecule, or (ii) comprises a sequence having at least 70% sequence identity with SEQ ID NO: 1, SEQ ID NO: 2, SEQ ID NO: 3, SEQ ID NO: 4, or SEQ ID NO: 5.

2. The method of claim 1, wherein said nucleic acid comprises a sequence with no more than a 4 nucleotide difference from SEQ ID NO: 1, SEQ ID NO: 2, SEQ ID NO: 3, SEQ ID NO: 4, or SEQ ID NO: 5.

3. The method of claim 1, wherein said nucleic acid comprises a sequence having 100% sequence identity with SEQ ID NO: 1, SEQ ID NO: 2, SEQ ID NO: 3, SEQ ID NO: 4, or SEQ ID NO: 5.

4. The method of claim 1, wherein said cell is a cancer cell.

5. The method of claim 1, wherein said cell is a human breast cancer cell.

6. The method of claim 1, wherein said RNA molecule is selected from miRNA-150, miRNA-204, miRNA-449, miRNA-450-1, and miRNA-508.

7. The method of claim 1, wherein said nucleic acid is at least 12 nucleotides in length.

8. The method of claim 1, wherein said nucleic acid is 12 to 30 nucleotides in length.

9. The method of claim 1, wherein said nucleic acid comprises at least one modified internucleotide linkage selected from the group consisting of phosphoroamidate, phosphorothiate, phosphorodithioate, boranophosphate, alkylphosphonate, and methylinemethylimino.

10. The method of claim 1, wherein said nucleic acid comprises a modified nucleic acid unit selected from the group consisting of locked nucleic acid unit, 2′-O-methyl ribonucleic acid unit, 2′O-methoxy-ethyl ribonucleic acid unit, 2′alkyl ribonucleic acid unit, 2′amine ribonucleic acid unit, peptide nucleic acid unit, 2′fluoro-ribo nucleic acid unit, morpholino nucleic acid unit, cyclohexane nucleic acid unit, or a tricyclonucleic acid unit.

11. The method of claim 1, wherein said nucleic acid is a locked nucleic acid, a 2′-O-methyl ribonucleic acid, or a mixed nucleic acid-locked nucleic acid.

12. The method of claim 1, wherein decreasing cell viability comprises increasing apoptosis.

13. The method of claim 1, wherein decreasing cell viability comprises decreasing cell viability.

14. The method of claim 1, wherein introducing the nucleic acid results in increased FKHR in the nucleus of a target cell.

15. A method of treating cancer in a subject in need thereof, said method comprising administering to said subject an effective amount of a nucleic acid hybridizable to an RNA molecule, wherein:
(a) said RNA molecule is selected from the group consisting of miRNA-150, miRNA-204, miRNA-449, miRNA-450-1, miRNA-508, and precursors thereof; and
(b) said nucleic acid (i) hybridizes under stringent conditions to said RNA molecule, or (ii) comprises a sequence having at least 70% sequence identity with SEQ ID NO: 1, SEQ ID NO: 2, SEQ ID NO: 3, SEQ ID NO: 4, or SEQ ID NO: 5.

16. The method of claim 15, wherein said nucleic acid comprises a sequence with no more than a 4 nucleotide difference from SEQ ID NO: 1, SEQ ID NO: 2, SEQ ID NO: 3, SEQ ID NO: 4, or SEQ ID NO: 5.

17. The method of claim 15, wherein said nucleic acid comprises a sequence having 100% sequence identity with SEQ ID NO: 1, SEQ ID NO: 2, SEQ ID NO: 3, SEQ ID NO: 4, or SEQ ID NO: 5.

18. The method of claim 15, wherein said cancer is a human breast cancer.

19. The method of claim 15, wherein said RNA molecule is selected from miRNA-449, miRNA-450-1, miRNA-508, and precursors thereof.

20. The method of claim 15, wherein said RNA molecule is selected from miRNA-150, miRNA-204, miRNA-449, miRNA-450-1, and miRNA-508.

21. The method of claim 15, wherein said RNA molecule is selected from miRNA-449, miRNA-450-1, and miRNA-508.

22. The method of claim 15, wherein said nucleic acid is at least 12 nucleotides in length.

23. The method of claim 15, wherein said nucleic acid is 12 to 30 nucleotides in length.

24. The method of claim 15, wherein said nucleic acid comprises at least one modified internucleotide linkage selected from the group consisting of phosphoroamidate, phosphorothiate, phosphorodithioate, boranophosphate, alkylphosphonate, and methylinemethylimino.

25. The method of claim 15, wherein said nucleic acid comprises a modified nucleic acid unit selected from the group consisting of locked nucleic acid unit, 2′-O-methyl ribonucleic acid unit, 2′O-methoxy-ethyl ribonucleic acid unit, 2′alkyl ribonucleic acid unit, 2′amine ribonucleic acid unit, peptide nucleic acid unit, 2′fluoro-ribo nucleic acid unit, morpholino nucleic acid unit, cyclohexane nucleic acid unit, or a tricyclonucleic acid unit.

26. The method of claim 15, wherein said nucleic acid is a locked nucleic acid, a 2′-O-methyl ribonucleic acid, or a mixed nucleic acid-locked nucleic acid.

27. A nucleic acid comprising at least 50% locked nucleic acid units, wherein said nucleic acid (i) hybridizes under stringent conditions to an RNA molecule, or (ii) comprises a sequence having at least 70% sequence identity with SEQ ID NO: 1, SEQ ID NO: 2, SEQ ID NO: 3, SEQ ID NO: 4, or SEQ ID NO: 5, wherein said RNA molecule is selected from the group consisting of miRNA-150, miRNA-204, miRNA-449, miRNA-450-1, miRNA-508, and precursors thereof.

28. The nucleic acid of claim 27 further comprising at least one cholesterol moiety.

29. The nucleic acid of claim 28, wherein the at least one cholesterol moiety is linked to the 5′ or 3′, or 5′ and 3′, terminus of the nucleic acid.

30. The nucleic acid of claim 27, wherein the nucleic acid comprises at least 70% locked nucleic acid units.

31. The nucleic acid of claim 27, wherein the nucleic acid comprises at least 90% locked nucleic acid units.
